SEQUENCE LISTING (56 sequences)

SEQ ID NO: 1 - DNA, 30 bp, synthetic construct
  source 1..30: mol_type = other DNA
  misc_feature 11..30: note = alpha-L-threofuranosyl nucleic acids
ggataccaccgagacgacacacaatagaga

SEQ ID NO: 2 - DNA, 30 bp, synthetic construct; identical to SEQ ID NO: 1
  source 1..30: mol_type = other DNA
  misc_feature 11..30: note = alpha-L-threofuranosylnucleic acids
  modified_base 25: mod_base = OTHER; note = OTHER indicates a base modified with a benzyl moiety at position C-5

SEQ ID NO: 3 - DNA, 30 bp, synthetic construct; identical to SEQ ID NO: 1
  source 1..30: mol_type = other DNA
  misc_feature 11..30: note = alpha-L-threofuranosyl nucleic acids
  modified_base 25: mod_base = OTHER; note = OTHER indicates base modified with an indole moiety at position C-5

SEQ ID NO: 4 - DNA, 40 bp, synthetic construct
  source 1..40: mol_type = other DNA
  misc_feature 1..40: note = alpha-L-threofuranosyl nucleic acids
tggcgaggggtagtgagctgacggatagggctagtggaac

SEQ ID NO: 5 - DNA, 40 bp, synthetic construct
  source 1..40: mol_type = other DNA
  misc_feature 1..40: note = alpha-L-threofuranosyl nucleic acids
cgtgcacgatctagccagtgctcgcttgaaatgggcgcag

SEQ ID NO: 6 - DNA, 40 bp, synthetic construct
  source 1..40: mol_type = other DNA
  misc_feature 1..40: note = alpha-L-threofuranosyl nucleic acids
  modified_base 1: mod_base = OTHER; note = OTHER indicates a base modified with a benzyl moiety at position C-5
  modified_base 3: mod_base = OTHER; note = OTHER indicates a base modified with a benzyl moiety at position C-5
  modified_base 4: mod_base = OTHER; note = OTHER indicates a base modified with a benzyl moiety at position C-5
  modified_base 10: mod_base = OTHER; note = OTHER indicates a base modified with a benzyl moiety at position C-5
  modified_base 22: mod_base = OTHER; note = OTHER indicates a base modified with a benzyl moiety at position C-5
  modified_base 24: mod_base = OTHER; note = OTHER indicates a base modified with a benzyl moiety at position C-5
  modified_base 29: mod_base = OTHER; note = OTHER indicates a base modified with a benzyl moiety at position C-5
  modified_base 37: mod_base = OTHER; note = OTHER indicates a base modified with a benzyl moiety at position C-5
tattggaactcccgagcaggctgtcccatggaagactaca

SEQ ID NO: 7 - DNA, 40 bp, synthetic construct
  source 1..40: mol_type = other DNA
  misc_feature 1..40: note = alpha-L-threofuranosyl nucleic acids
  modified_base 5: mod_base = OTHER; note = OTHER indicates a base modified with a benzyl moiety at position C-5
  modified_base 6: mod_base = OTHER; note = OTHER indicates a base modified with a benzyl moiety at position C-5
  modified_base 8: mod_base = OTHER; note = OTHER indicates a base modified with a benzyl moiety at position C-5
  modified_base 13: mod_base = OTHER; note = OTHER indicates a base modified with a benzyl moiety at position C-5
  modified_base 15..16: mod_base = OTHER; note = OTHER indicates a base modified with a benzyl moiety at position C-5
  modified_base 18: mod_base = OTHER; note = OTHER indicates a base modified with a benzyl moiety at position C-5
  modified_base 20: mod_base = OTHER; note = OTHER indicates a base modified with a benzyl moiety at position C-5
  modified_base 31: mod_base = OTHER; note = OTHER indicates a base modified with a benzyl moiety at position C-5
  modified_base 40: mod_base = OTHER; note = OTHER indicates a base modified with a benzyl moiety
aggcttatgaaatcttatgtgaaccgggcatacgggcgat

SEQ ID NO: 8 - DNA, 40 bp, synthetic construct
  source 1..40: mol_type = other DNA
  misc_feature 1..40: note = alpha-L-threofuranosyl nucleic acids
  modified_base 6: mod_base = OTHER; note = OTHER indicates a base modified with a benzyl moiety at position C-5
  modified_base 9: mod_base = OTHER; note = OTHER indicates a base modified with a benzyl moiety at position C-5
  modified_base 12: mod_base = OTHER; note = OTHER indicates a base modified with a benzyl moiety at position C-5
  modified_base 15..16: mod_base = OTHER; note = OTHER indicates a base modified with a benzyl moiety at position C-5
  modified_base 23: mod_base = OTHER; note = OTHER indicates a base modified with a benzyl moiety at position C-5
  modified_base 25: mod_base = OTHER; note = OTHER indicates a base modified with a benzyl moiety at position C-5
  modified_base 31..32: mod_base = OTHER; note = OTHER indicates a base modified with a benzyl moiety at position C-5
  modified_base 38: mod_base = OTHER; note = OTHER indicates a base modified with a benzyl moiety at position C-5
  modified_base 40: mod_base = OTHER; note = OTHER indicates a base modified with a benzyl moiety at position C-5
gagagtcatagtagttcacgggtctgcagcttacgcatgt

SEQ ID NO: 9 - DNA, 40 bp, synthetic construct
  source 1..40: mol_type = other DNA
  misc_feature 1..40: note = alpha-L-threofuranosyl nucleic acids
  modified_base 1: mod_base = OTHER; note = OTHER indicates a base modified with a benzyl moiety at position C-5
  modified_base 3: mod_base = OTHER; note = OTHER indicates a base modified with a benzyl moiety at position C-5
  modified_base 10: mod_base = OTHER; note = OTHER indicates a base modified with a benzyl moiety at position C-5
  modified_base 12: mod_base = OTHER; note = OTHER indicates a base modified with a benzyl moiety at position C-5
  modified_base 19: mod_base = OTHER; note = OTHER indicates a base modified with a benzyl moiety at position C-5
  modified_base 22: mod_base = OTHER; note = OTHER indicates a base modified with a benzyl moiety at position C-5
  modified_base 35: mod_base = OTHER; note = OTHER indicates a base modified with a benzyl moiety at position C-5
  modified_base 38: mod_base = OTHER; note = OTHER indicates a base modified with a benzyl moiety at position C-5
tgtacgccatataggcagtcatcgagcaacgaagtaatgg

SEQ ID NO: 10 - DNA, 40 bp, synthetic construct
  source 1..40: mol_type = other DNA
  misc_feature 1..40: note = alpha-L-threofuranosyl nucleic acids
  modified_base 5: mod_base = OTHER; note = OTHER indicates a base modified with a benzyl moiety at position C-5
  modified_base 10..11: mod_base = OTHER; note = OTHER indicates a base modified with a benzyl moiety at position C-5
  modified_base 15: mod_base = OTHER; note = OTHER indicates a base modified with a benzyl moiety at position C-5
  modified_base 19: mod_base = OTHER; note = OTHER indicates a base modified with a benzyl moiety at position C-5
  modified_base 22: mod_base = OTHER; note = OTHER indicates a base modified with a benzyl moiety at position C-5
  modified_base 27..28: mod_base = OTHER; note = OTHER indicates a base modified with a benzyl moiety at position C-5
  modified_base 33: mod_base = OTHER; note = OTHER indicates a base modified with a benzyl moiety at position C-5
  modified_base 35..37: mod_base = OTHER; note = OTHER indicates a base modified with a benzyl moiety at position C-5
acgatggacttgggtagctagtcaggttaggctgtttgga

SEQ ID NO: 11 - DNA, 40 bp, synthetic construct
  source 1..40: mol_type = other DNA
  misc_feature 1..40: note = alpha-L-threofuranosyl nucleic acids
  modified_base 6..7: mod_base = OTHER; note = OTHER indicates a base modified with a benzyl moiety at position C-5
  modified_base 10: mod_base = OTHER; note = OTHER indicates a base modified with a benzyl moiety at position C-5
  modified_base 16: mod_base = OTHER; note = OTHER indicates a base modified with a benzyl moiety at position C-5
  modified_base 18..19: mod_base = OTHER; note = OTHER indicates a base modified with a benzyl moiety at position C-5
  modified_base 26: mod_base = OTHER; note = OTHER indicates a base modified with a benzyl moiety at position C-5
  modified_base 35: mod_base = OTHER; note = OTHER indicates a base modified with a benzyl moiety at position C-5
  modified_base 39: mod_base = OTHER; note = OTHER indicates a base modified with a benzyl moiety at position C-5
agaacttcgtcagcgtcttaccgagtccaaggactggcta

SEQ ID NO: 12 - DNA, 40 bp, synthetic construct
  source 1..40: mol_type = other DNA
  misc_feature 1..40: note = alpha-L-threofuranosyl nucleic acids
  modified_base 2: mod_base = OTHER; note = OTHER indicates a base modified with a benzyl moiety at position C-5
  modified_base 7..8: mod_base = OTHER; note = OTHER indicates a base modified with a benzyl moiety at position C-5
  modified_base 10: mod_base = OTHER; note = OTHER indicates a base modified with a benzyl moiety at position C-5
  modified_base 15..16: mod_base = OTHER; note = OTHER indicates a base modified with a benzyl moiety at position C-5
  modified_base 22: mod_base = OTHER; note = OTHER indicates a base modified with a benzyl moiety at position C-5
  modified_base 25: mod_base = OTHER; note = OTHER indicates a base modified with a benzyl moiety at position C-5
  modified_base 33: mod_base = OTHER; note = OTHER indicates a base modified with a benzyl moiety at position C-5
  modified_base 38..39: mod_base = OTHER; note = OTHER indicates a base modified with a benzyl moiety at position C-5
atcgagttgtgacgttaacgataatagcccgctccgattc

SEQ ID NO: 13 - DNA, 40 bp, synthetic construct
  source 1..40: mol_type = other DNA
  misc_feature 1..40: note = alpha-L-threofuranosyl nucleic acids
  modified_base 1: mod_base = OTHER; note = OTHER indicates a base modified with a benzyl moiety at position C-5
  modified_base 5..6: mod_base = OTHER; note = OTHER indicates a base modified with a benzyl moiety at position C-5
  modified_base 12..13: mod_base = OTHER; note = OTHER indicates a base modified with a benzyl moiety at position C-5
  modified_base 18..19: mod_base = OTHER; note = OTHER indicates a base modified with a benzyl moiety at position C-5
  modified_base 22: mod_base = OTHER; note = OTHER indicates a base modified with a benzyl moiety at position C-5
  modified_base 26: mod_base = OTHER; note = OTHER indicates a base modified with a benzyl moiety at position C-5
  modified_base 28: mod_base = OTHER; note = OTHER indicates a base modified with a benzyl moiety at position C-5
  modified_base 30: mod_base = OTHER; note = OTHER indicates a base modified with a benzyl moiety at position C-5
  modified_base 39: mod_base = OTHER; note = OTHER indicates a base modified with a benzyl moiety at position C-5
tacattacgcgttagcgttaatgggtatgtccggacagtg

SEQ ID NO: 14 - DNA, 40 bp, synthetic construct
  source 1..40: mol_type = other DNA
  misc_feature 1..40: note = alpha-L-threofuranosyl nucleic acids
  modified_base 5: mod_base = OTHER; note = OTHER indicates a base modified with a benzyl moiety at position C-5
  modified_base 8: mod_base = OTHER; note = OTHER indicates a base modified with a benzyl moiety at position C-5
  modified_base 17: mod_base = OTHER; note = OTHER indicates a base modified with a benzyl moiety at position C-5
  modified_base 21: mod_base = OTHER; note = OTHER indicates a base modified with a benzyl moiety at position C-5
  modified_base 24..26: mod_base = OTHER; note = OTHER indicates a base modified with a benzyl moiety at position C-5
  modified_base 29: mod_base = OTHER; note = OTHER indicates a base modified with a benzyl moiety at position C-5
  modified_base 39: mod_base = OTHER; note = OTHER indicates a base modified with a benzyl moiety at position C-5
  modified_base 3: mod_base = OTHER; note = OTHER indicates a base modified with a benzyl moiety at position C-5
ggtgtaataaggagagtacgtcgtttggtgggaccgggta

SEQ ID NO: 15 - DNA, 40 bp, synthetic construct
  source 1..40: mol_type = other DNA
  misc_feature 1..40: note = alpha-L-threofuranosyl nucleic acids
  modified_base 1..2: mod_base = OTHER; note = OTHER indicates a base modified with a indole moiety at position C-5
  modified_base 10: mod_base = OTHER; note = OTHER indicates a base modified with a indole moiety at position C-5
  modified_base 15: mod_base = OTHER; note = OTHER indicates a base modified with a indole moiety at position C-5
  modified_base 17: mod_base = OTHER; note = OTHER indicates a base modified with a indole moiety at position C-5
  modified_base 21: mod_base = OTHER; note = OTHER indicates a base modified with a indole moiety at position C-5
  modified_base 26..27: mod_base = OTHER; note = OTHER indicates a base modified with a indole moiety at position C-5
  modified_base 30..33: mod_base = OTHER; note = OTHER indicates a base modified with a indole moiety at position C-5
  modified_base 35: mod_base = OTHER; note = OTHER indicates a base modified with a indole moiety at position C-5
  modified_base 38..40: mod_base = OTHER; note = OTHER indicates a base modified with a indole moiety at position C-5
ttggcgagatcccctatacgtgagcttccttttatccttt

SEQ ID NO: 16 - DNA, 40 bp, synthetic construct
  source 1..40: mol_type = other DNA
  modified_base 9: mod_base = OTHER; note = OTHER indicates a base modified with a indole moiety at position C-5
  modified_base 14..16: mod_base = OTHER; note = OTHER indicates a base modified with a indole moiety at position C-5
  modified_base 28..29: mod_base = OTHER; note = OTHER indicates a base modified with a indole moiety at position C-5
  modified_base 31..32: mod_base = OTHER; note = OTHER indicates a base modified with a indole moiety at position C-5
  modified_base 36..38: mod_base = OTHER; note = OTHER indicates a base modified with a indole moiety at position C-5
  misc_feature 1..40: note = alpha-L-threofuranosyl nucleic acid
  modified_base 3: mod_base = OTHER; note = OTHER indicates a base modified with a indole moiety at position C-5
cgtacaactccggtttcgggcggcccgttcttcagtttga

SEQ ID NO: 17 - DNA, 40 bp, synthetic construct
  source 1..40: mol_type = other DNA
  misc_feature 1..40: note = alpha-L-threofuranosyl nucleic acids
  modified_base 19: mod_base = OTHER; note = OTHER indicates a base modified with a indole moiety at position C-5
  modified_base 26..27: mod_base = OTHER; note = OTHER indicates a base modified with a indole moiety at position C-5
  modified_base 30: mod_base = OTHER; note = OTHER indicates a base modified with a indole moiety at position C-5
  modified_base 35: mod_base = OTHER; note = OTHER indicates a base modified with a indole moiety at position C-5
  modified_base 17: mod_base = OTHER; note = OTHER indicates a base modified with a indole moiety at position C-5
taaggaaaggggagcgggtaggaggttcgtcgggtgcgac

SEQ ID NO: 18 - DNA, 40 bp, synthetic construct
  source 1..40: mol_type = other DNA
  misc_feature 1..40: note = alpha-L-threofuranosyl nucleic acids
  modified_base 2..3: mod_base = OTHER; note = OTHER indicates a base modified with a indole moiety at position C-5
  modified_base 5: mod_base = OTHER; note = OTHER indicates a base modified with a indole moiety at position C-5
  modified_base 12: mod_base = OTHER; note = OTHER indicates a base modified with a indole moiety at position C-5
  modified_base 14: mod_base = OTHER; note = OTHER indicates a base modified with a indole moiety at position C-5
  modified_base 18: mod_base = OTHER; note = OTHER indicates a base modified with a indole moiety at position C-5
  modified_base 25: mod_base = OTHER; note = OTHER indicates a base modified with a indole moiety at position C-5
  modified_base 29: mod_base = OTHER; note = OTHER indicates a base modified with a indole moiety at position C-5
  modified_base 34: mod_base = OTHER; note = OTHER indicates a base modified with a indole moiety at position C-5
attgtgcgaagtatacatagcagatgcgtgacataacaag

SEQ ID NO: 19 - DNA, 40 bp, synthetic construct
  source 1..40: mol_type = other DNA
  misc_feature 1..40: note = alpha-L-threofuranosyl nucleic acids
  modified_base 7..8: mod_base = OTHER; note = OTHER indicates a base modified with a indole moiety at position C-5
  modified_base 14..15: mod_base = OTHER; note = OTHER indicates a base modified with a indole moiety at position C-5
  modified_base 17: mod_base = OTHER; note = OTHER indicates a base modified with a indole moiety at position C-5
  modified_base 20: mod_base = OTHER; note = OTHER indicates a base modified with a indole moiety at position C-5
  modified_base 22: mod_base = OTHER; note = OTHER indicates a base modified with a indole moiety at position C-5
  modified_base 25: mod_base = OTHER; note = OTHER indicates a base modified with a indole moiety at position C-5
  modified_base 28: mod_base = OTHER; note = OTHER indicates a base modified with a indole moiety at position C-5
  modified_base 33: mod_base = OTHER; note = OTHER indicates a base modified with a indole moiety at position C-5
  modified_base 39: mod_base = OTHER; note = OTHER indicates a base modified with a indole moiety at position C-5
  modified_base 5: mod_base = OTHER; note = OTHER indicates a base modified with a indole moiety at position C-5
aaactgttgggacttgtaatgtcgtcatcagctaagagtc

SEQ ID NO: 20 - DNA, 40 bp, synthetic construct
  source 1..40: mol_type = other DNA
  misc_feature 1..40: note = alpha-L-threofuranosyl nucleic acids
  modified_base 1..3: mod_base = OTHER; note = OTHER indicates a base modified with a indole moiety at position C-5
  modified_base 5: mod_base = OTHER; note = OTHER indicates a base modified with a indole moiety at position C-5
  modified_base 7: mod_base = OTHER; note = OTHER indicates a base modified with a indole moiety at position C-5
  modified_base 11: mod_base = OTHER; note = OTHER indicates a base modified with a indole moiety at position C-5
  modified_base 13: mod_base = OTHER; note = OTHER indicates a base modified with a indole moiety at position C-5
  modified_base 20: mod_base = OTHER; note = OTHER indicates a base modified with a indole moiety at position C-5
  modified_base 27: mod_base = OTHER; note = OTHER indicates a base modified with a indole moiety at position C-5
  modified_base 29: mod_base = OTHER; note = OTHER indicates a base modified with a indole moiety at position C-5
tttctatgcatctcggggatgcgggatataaaagagagac

SEQ ID NO: 21 - DNA, 40 bp, synthetic construct
  source 1..40: mol_type = other DNA
  misc_feature 1..40: note = alpha-L-threofuranosyl nucleic acids
gcggggggtgttgtgtaactgttttttgccggaaagacgc

SEQ ID NO: 22 - DNA, 40 bp, synthetic construct
  source 1..40: mol_type = other DNA
  misc_feature 1..40: note = alpha-L-threofuranosyl nucleic acids
gacgctatgactggtatggtgtgtgagcttcgaggtctga

SEQ ID NO: 23 - DNA, 40 bp, synthetic construct
  source 1..40: mol_type = other DNA
  misc_feature 1..40: note = alpha-L-threofuranosyl nucleic acids
cgcggcgttgcgtagcgccaatcctgcacagaggcgggcg

SEQ ID NO: 24 - DNA, 40 bp, synthetic construct
  source 1..40: mol_type = other DNA
  misc_feature 1..40: note = alpha-L-threofuranosyl nucleic acids
  modified_base 1: mod_base = OTHER; note = OTHER indicates a base modified with benzyl moeity at position C-5
  modified_base 8: mod_base = OTHER; note = OTHER indicates a base modified with benzyl moeity at position C-5
  modified_base 17: mod_base = OTHER; note = OTHER indicates a base modified with benzyl moeity at position C-5
  modified_base 30..31: mod_base = OTHER; note = OTHER indicates a base modified with benzyl moeity at position C-5
  modified_base 33..36: mod_base = OTHER; note = OTHER indicates a base modified with benzyl moeity at position C-5
  modified_base 40: mod_base = OTHER; note = OTHER indicates a base modified with benzyl moeity at position C-5
tgacacctggcacgaatgcgcagacgacgttgttttgcgt

SEQ ID NO: 25 - DNA, 40 bp, synthetic construct
  source 1..40: mol_type = other DNA
  misc_feature 1..40: note = alpha-L-threofuranosyl nucleic acids
  modified_base 3..6: mod_base = OTHER; note = OTHER indicates a base modified with benzyl moeity at position C-5
  modified_base 9..10: mod_base = OTHER; note = OTHER indicates a base modified with benzyl moeity at position C-5
  modified_base 15..16: mod_base = OTHER; note = OTHER indicates a base modified with benzyl moeity at position C-5
  modified_base 24..26: mod_base = OTHER; note = OTHER indicates a base modified with benzyl moeity at position C-5
  modified_base 29: mod_base = OTHER; note = OTHER indicates a base modified with benzyl moeity at position C-5
  modified_base 34: mod_base = OTHER; note = OTHER indicates a base modified with benzyl moeity at position C-5
  modified_base 37: mod_base = OTHER; note = OTHER indicates a base modified with benzyl moeity at position C-5
  modified_base 39: mod_base = OTHER; note = OTHER indicates a base modified with benzyl moeity at position C-5
acttttgcttgagattcggcgcatttcgtgcagtgatgta

SEQ ID NO: 26 - DNA, 40 bp, synthetic construct
  source 1..40: mol_type = other DNA
  misc_feature 1..40: note = alpha-L-threofuranosyl nucleic acids
  modified_base 2..3: mod_base = OTHER; note = OTHER indicates a base modified with benzyl moeity at position C-5
  modified_base 7: mod_base = OTHER; note = OTHER indicates a base modified with benzyl moeity at position C-5
  modified_base 13..15: mod_base = OTHER; note = OTHER indicates a base modified with benzyl moeity at position C-5
  modified_base 21..22: mod_base = OTHER; note = OTHER indicates a base modified with benzyl moeity at position C-5
  modified_base 25: mod_base = OTHER; note = OTHER indicates a base modified with benzyl moeity at position C-5
  modified_base 33..34: mod_base = OTHER; note = OTHER indicates a base modified with benzyl moeity at position C-5
cttaggtcggcctttgaccattcgtgcaaaacttagcgaa

SEQ ID NO: 27 - DNA, 40 bp, synthetic construct
  source 1..40: mol_type = other DNA
  misc_feature 1..40: note = alpha-L-threofuranosyl nucleic acids
  modified_base 4: mod_base = OTHER; note = OTHER indicates a base modified with benzyl moeity at position C-5
  modified_base 9..10: mod_base = OTHER; note = OTHER indicates a base modified with benzyl moeity at position C-5
  modified_base 23: mod_base = OTHER; note = OTHER indicates a base modified with benzyl moeity at position C-5
  modified_base 26: mod_base = OTHER; note = OTHER indicates a base modified with benzyl moeity at position C-5
  modified_base 28: mod_base = OTHER; note = OTHER indicates a base modified with benzyl moeity at position C-5
gaatgcgcttgagcgcgcccgatgatatagcgaaccaagc

SEQ ID NO: 28 - DNA, 40 bp, synthetic construct
  source 1..40: mol_type = other DNA
  misc_feature 1..40: note = alpha-L-threofuranosyl nucleic acids
  modified_base 1..2: mod_base = OTHER; note = OTHER indicates a base modified with benzyl moeity at position C-5
  modified_base 5..8: mod_base = OTHER; note = OTHER indicates a base modified with benzyl moeity at position C-5
  modified_base 13: mod_base = OTHER; note = OTHER indicates a base modified with benzyl moeity at position C-5
  modified_base 15..17: mod_base = OTHER; note = OTHER indicates a base modified with benzyl moeity at position C-5
  modified_base 19: mod_base = OTHER; note = OTHER indicates a base modified with benzyl moeity at position C-5
  modified_base 24..25: mod_base = OTHER; note = OTHER indicates a base modified with benzyl moeity at position C-5
  modified_base 28: mod_base = OTHER; note = OTHER indicates a base modified with benzyl moeity at position C-5
  modified_base 32: mod_base = OTHER; note = OTHER indicates a base modified with benzyl moeity at position C-5
  modified_base 39: mod_base = OTHER; note = OTHER indicates a base modified with benzyl moeity at position C-5
ttaattttgagatctttgtcgagttgctcggtgaaaagtc

SEQ ID NO: 29 - DNA, 40 bp, synthetic construct
  source 1..40: mol_type = other DNA
  misc_feature 1..40: note = alpha-L-threofuranosyl nucleic acids
  modified_base 9: mod_base = OTHER; note = OTHER indicates a base modified with benzyl moeity at position C-5
  modified_base 12: mod_base = OTHER; note = OTHER indicates a base modified with benzyl moeity at position C-5
  modified_base 15: mod_base = OTHER; note = OTHER indicates a base modified with benzyl moeity at position C-5
  modified_base 22..24: mod_base = OTHER; note = OTHER indicates a base modified with benzyl moeity at position C-5
  modified_base 28..32: mod_base = OTHER; note = OTHER indicates a base modified with benzyl moeity at position C-5
  modified_base 35..37: mod_base = OTHER; note = OTHER indicates a base modified with benzyl moeity at position C-5
agagcaagtcatgatcgcgagtttagctttttcatttccc

SEQ ID NO: 30 - DNA, 40 bp, synthetic construct
  source 1..40: mol_type = other DNA
  misc_feature 1..40: note = alpha-L-threofuranosyl nucleic acids
  modified_base 6: mod_base = OTHER; note = OTHER indicates a base modified with indole moeity at position C-5
  modified_base 8: mod_base = OTHER; note = OTHER indicates a base modified with indole moeity at position C-5
  modified_base 12..13: mod_base = OTHER; note = OTHER indicates a base modified with indole moeity
  modified_base 15: mod_base = OTHER; note = OTHER indicates a base modified with indole moeity at position C-5
  modified_base 18: mod_base = OTHER; note = OTHER indicates a base modified with indole moeity at position C-5
  modified_base 24..25: mod_base = OTHER; note = OTHER indicates a base modified with indole moeity at position C-5
  modified_base 28..29: mod_base = OTHER; note = OTHER indicates a base modified with indole moeity at position C-5
  modified_base 31: mod_base = OTHER; note = OTHER indicates a base modified with indole moeity at position C-5
  modified_base 33: mod_base = OTHER; note = OTHER indicates a base modified with indole moeity at position C-5
  modified_base 40: mod_base = OTHER; note = OTHER indicates a base modified with indole moeity at position C-5
ggcactatcgattatcgtgcgcattggttgtgtccaggct

SEQ ID NO: 31 - DNA, 40 bp, synthetic construct
  source 1..40: mol_type = other DNA
  misc_feature 1..40: note = alpha-L-threofuranosyl nucleic acids
  modified_base 1: mod_base = OTHER; note = OTHER indicates a base modified with indole moeity at position C-5
  modified_base 6: mod_base = OTHER; note = OTHER indicates a base modified with indole moeity at position C-5
  modified_base 8: mod_base = OTHER; note = OTHER indicates a base modified with indole moeity at position C-5
  modified_base 10..11: mod_base = OTHER; note = OTHER indicates a base modified with indole moeity at position C-5
  modified_base 13: mod_base = OTHER; note = OTHER indicates a base modified with indole moeity at position C-5
  modified_base 18..19: mod_base = OTHER; note = OTHER indicates a base modified with indole moeity at position C-5
  modified_base 21: mod_base = OTHER; note = OTHER indicates a base modified with indole moeity at position C-5
  modified_base 26: mod_base = OTHER; note = OTHER indicates a base modified with indole moeity at position C-5
tcacgtctgttgtacacttgtgcaatgcaaggacggaaaa

SEQ ID NO: 32 - DNA, 40 bp, synthetic construct
  source 1..40: mol_type = other DNA
  misc_feature 1..40: note = alpha-L-threofuranosyl nucleic acids
  modified_base 6: mod_base = OTHER; note = OTHER indicates a base modified with indole moeity at position C-5
  modified_base 15..16: mod_base = OTHER; note = OTHER indicates a base modified with indole moeity at position C-5
  modified_base 18: mod_base = OTHER; note = OTHER indicates a base modified with indole moeity at position C-5
  modified_base 21: mod_base = OTHER; note = OTHER indicates a base modified with indole moeity at position C-5
  modified_base 24..26: mod_base = OTHER; note = OTHER indicates a base modified with indole moeity at position C-5
  modified_base 31: mod_base = OTHER; note = OTHER indicates a base modified with indole moeity at position C-5
  modified_base 33..35: mod_base = OTHER; note = OTHER indicates a base modified with indole moeity at position C-5
  modified_base 38: mod_base = OTHER; note = OTHER indicates a base modified with indole moeity at position C-5
acgagtaggccgcattatgatgatttaaactgtttcatgc

SEQ ID NO: 33 - DNA, 40 bp, synthetic construct
  source 1..40: mol_type = other DNA
  misc_feature 1..40: note = alpha-L-threofuranosyl nucleic acids
gcggccagatccccgatatgttaacgtccttttatgcggg

SEQ ID NO: 34 - DNA, 40 bp, synthetic construct
  source 1..40: mol_type = other DNA
  misc_feature 1..40: note = alpha-L-threofuranosyl nucleic acids
ttagtattattatgggtgcgtgagtttccaagaagcgcgt

SEQ ID NO: 35 - DNA, 20 bp, synthetic construct
  source 1..20: mol_type = other DNA
ggatcgtcagtgcattgaga

SEQ ID NO: 36 - DNA, 52 bp, synthetic construct
  source 1..52: mol_type = other DNA
  misc_feature 32^33: note = spacer
aacaaacaaacaaacaaacaaacaaacaaacaggatcgtcagtgcattgaga

SEQ ID NO: 37 - DNA, 20 bp, synthetic construct
  source 1..20: mol_type = other DNA
gtccccttggggataccacc

SEQ ID NO: 38 - DNA, 20 bp, synthetic construct
  source 1..20: mol_type = other DNA
ttctcatgtttgacagctta

SEQ ID NO: 39 - DNA, 52 bp, synthetic construct
  source 1..52: mol_type = other DNA
  misc_feature 32^33: note = spacer
aacaaacaaacaaacaaacaaacaaacaaacattctcatgtttgacagctta

SEQ ID NO: 40 - DNA, 20 bp, synthetic construct
  source 1..20: mol_type = other DNA
tcatcgataagctttaatgc

SEQ ID NO: 41 - DNA, 52 bp, synthetic construct
  source 1..52: mol_type = other DNA
  misc_feature 32^33: note = spacer
aacaaacaaacaaacaaacaaacaaacaaacatcatcgataagctttaatgc

SEQ ID NO: 42 - DNA, 20 bp, synthetic construct
  source 1..20: mol_type = other DNA
ttgctaacgcagtcaggcac

SEQ ID NO: 43 - DNA, 52 bp, synthetic construct
  source 1..52: mol_type = other DNA
  misc_feature 32^33: note = spacer
aacaaacaaacaaacaaacaaacaaacaaacattgctaacgcagtcaggcac

SEQ ID NO: 44 - DNA, 60 bp, synthetic construct
  source 1..60: mol_type = other DNA
actacgtacccacaacctcggccgtaccacggtacgtagtgtccccttggggataccacc

SEQ ID NO: 45 - DNA, 50 bp, synthetic construct
  source 1..50: mol_type = other DNA
ggtggtatccccaaggggacactacgtaccgtggtacggccgaggttgtg

SEQ ID NO: 46 - DNA, 50 bp, synthetic construct
  source 1..50: mol_type = other DNA
tctctattgtgagtcgtctaggtggtatcctttttttttttttttttttt

SEQ ID NO: 47 - DNA, 86 bp, synthetic construct
  source 1..86: mol_type = other DNA
ttctcatgtttgacagcttaattcctnnnnnnnnnnnnnnnnnnnnnnnnnnnnnnnnnnnnnnnnggtggtatccccaaggggac

SEQ ID NO: 48 - DNA, 86 bp, synthetic construct
  source 1..86: mol_type = other DNA
tcatcgataagctttaatgccactcannnnnnnnnnnnnnnnnnnnnnnnnnnnnnnnnnnnnnnnggtggtatccccaaggggac

SEQ ID NO: 49 - DNA, 86 bp, synthetic construct
  source 1..86: mol_type = other DNA
ttgctaacgcagtcaggcacccaacannnnnnnnnnnnnnnnnnnnnnnnnnnnnnnnnnnnnnnnggtggtatccccaaggggac

SEQ ID NO: 50 - DNA, 86 bp, synthetic construct
  source 1..86: mol_type = other DNA
  misc_feature 21..26: note = alpha-L-threofuranosyl nucleic acids
ggatcgtcagtgcattgagaattcctnnnnnnnnnnnnnnnnnnnnnnnnnnnnnnnnnnnnnnnnggtggtatccccaaggggac

SEQ ID NO: 51 - DNA, 86 bp, synthetic construct
  source 1..86: mol_type = other DNA
  misc_feature 21..26: note = alpha-L-threofuranosyl nucleic acids
ggatcgtcagtgcattgagacactcannnnnnnnnnnnnnnnnnnnnnnnnnnnnnnnnnnnnnnnggtggtatccccaaggggac

SEQ ID NO: 52 - DNA, 86 bp, synthetic construct
  source 1..86: mol_type = other DNA
  misc_feature 21..26: note = alpha-L-threofuranosyl nucleic acids
ggatcgtcagtgcattgagaccaacannnnnnnnnnnnnnnnnnnnnnnnnnnnnnnnnnnnnnnnggtggtatccccaaggggac

SEQ ID NO: 53 - DNA, 86 bp, synthetic construct
  source 1..86: mol_type = other DNA
ttgctaacgcagtcaggcactggcctnnnnnnnnnnnnnnnnnnnnnnnnnnnnnnnnnnnnnnnnggtggtatccccaaggggac

SEQ ID NO: 54 - DNA, 10 bp, synthetic construct
  source 1..10: mol_type = other DNA
ggataccacc

SEQ ID NO: 55 - DNA, 29 bp, synthetic construct
  source 1..29: mol_type = other DNA
  misc_feature 11..29: note = alpha-L-threofuranosyl nucleic acids
ggataccacctagacgactcacaatgaga

SEQ ID NO: 56 - DNA, 30 bp, synthetic construct
  source 1..30: mol_type = other DNA
cctatggtggatctgctgagtgttatctct

(19) United States
(12) Patent Application Publication
(10) Pub. No.: US 2025/0092406 A1
(43) Pub. Date: Mar. 20, 2025

(54) FUNCTIONALLY-ENHANCED XNA

(71) Applicant: THE REGENTS OF THE UNIVERSITY OF CALIFORNIA, Oakland, CA (US)

(72) Inventor: John Chaput, Irvine, CA (US)

(21) Appl. No.: 18/293,292

(22) PCT Filed: Aug. 2, 2022
(86) PCT No.: PCT/US2022/039086

§ 371 Date: Jan. 29, 2024

Related U.S. Application Data
(60) Provisional application No. 63/227,489, filed on Jul. 30, 2021.

(51) Int. Cl. C12N 15/115

(52) U.S. Cl. CPC C12N 15/115; C12N 2310/16; C12N 2310/314; C12N 2310/323; C12N 2310/344

(57) ABSTRACT

Synthetic genetic polymers (XNAs) have the potential to transition aptamers from laboratory tools to therapeutic agents, but additional functionality is needed to compete with antibodies. The present invention features compositions and methods that utilizes a biologically stable artificial genetic system comprised of α-L-threofuranosyl nucleic acid (TNA). This system facilitates the production of backbone- and base-modified aptamers termed ‘threomers’ that function as high quality protein capture reagents.

### CROSS-REFERENCES TO RELATED APPLICATIONS

This application claims benefit of U.S. Provisional Application No. 63/227,489 filed Jul. 30, 2021, the specification of which is incorporated herein in their entirety by reference.

### REFERENCE TO A SEQUENCE LISTING

Applicant asserts that the information recorded in the form of an Annex C/ST.26 file submitted under Rule 13ter.1 (a), entitled >>>UCI 21.14 PCT<<<, is identical to that forming part of the international application as filed. The content of the sequence listing is incorporated herein by reference in its entirety

### FIELD OF THE INVENTION

The present invention features compositions and methods for improving the activity and affinity of in vitro selected XNA aptamers as affinity reagents to protein targets.

### BACKGROUND OF THE INVENTION

The effectiveness of antibodies in diagnostic and therapeutic applications have inspired efforts to explore the chemical space of evolvable non-natural genetic systems in search of sequence-defined macromolecules that can recapitulate antibody binding by folding into shapes that recognize disease-associated proteins with high affinity and high specificity. Toward this goal, much attention has been given to the establishment of Darwinian evolution systems that allow for the isolation of artificial genetic polymers with nucleobase or sugar-phosphate backbone modifications that function with enhanced target binding affinity or elevated biological stability. However, despite many notable accomplishments, including the evolution of novel synthetic genetic polymers (XNAs) with backbone chemistries not found in nature and the creation of highly functionalized nucleic acid polymers (HFNAPs), the ability to generate affinity reagents that are both recalcitrant to biological nucleases and capable of recognizing their cognate protein target with low dissociation rate constants has proven elusive. Since slow-off rate binding is a hallmark of a high quality antibody, renewed efforts are needed to establish examples of biologically stable affinity reagents that mirror the binding properties of the best monoclonal antibodies.

### BRIEF SUMMARY OF THE INVENTION

It is an objective of the present invention to provide compositions and methods that allow for improvements in the activity and affinity of in vitro selected XNA (e.g., TNA) aptamers (e.g., threomers) as affinity reagents to protein targets, as specified in the independent claims. Embodiments of the invention are given in the dependent claims. Embodiments of the present invention can be freely combined with each other if they are not mutually exclusive.

Synthetic genetic polymers (XNAs) have the potential to transition aptamers from laboratory tools to therapeutic agents, but additional functionality is needed to compete with antibodies. Described herein is the evolution of a biologically stable artificial genetic system comprised of α-L-threofuranosyl nucleic acid (TNA) that facilitates the production of backbone- and base-modified aptamers termed ‘threomers’ that function as high quality protein capture reagents. Threomers were discovered against two prototypical protein targets implicated in human diseases through a combination of in vitro selection and next-generation sequencing using uracil nucleotides that are uniformly equipped with aromatic side chains commonly found in the paratope of antibody-antigen crystal structures. Kinetic measurements reveal that the side chain modifications are critical for generating threomers with slow off-rate binding kinetics. These findings expand the chemical space of evolvable non-natural genetic systems to include functional groups that enhance protein target binding by mimicking the structural properties of traditional antibodies.

In some embodiments, the present invention features base-modified xeno nucleic acid (XNA) nucleoside monomers. The base-modified XNA nucleoside monomers may comprise a synthetic, non-natural sugar (e.g., a threose or a hexose sugar), a pyrimidine nucleotide base comprising a chemical modification (e.g., at position C-5 of the nucleobase) bound to the sugar moiety (i.e., the synthetic non-natural sugar), and a phosphorus group also bound to the sugar moiety.

In other embodiments, the present invention features a based-modified threose nucleic acid (TNA) nucleoside monomer. The base-modified TNA nucleoside monomer may comprise a threose sugar, a pyrimidine nucleotide base comprising a chemical modification (e.g., at position C-5 of the nucleobase) bound to the sugar moiety (i.e., the threose sugar), and a phosphorus group also bound to the sugar moiety.

Additionally, the present invention may feature single-stranded oligonucleotide aptamer (e.g., threomers) for binding a target protein. The aptamer comprise one or more of the base-modified nucleoside monomers described herein. For example, the present invention may feature a single-stranded threose nucleic acid (TNA) aptamer (i.e., a threomer) for binding a target protein. The aptamer may comprise one or more of the base-modified TNA nucleoside monomers as described herein.

One of the unique and inventive technical features of the present invention is the use of a modified pyrimidine nucleotide base (e.g., a pyrimidine nucleotide base comprising a chemical modification, e.g., at position C-5 of the nucleobase). Without wishing to limit the invention to any theory or mechanism, it is believed that the technical feature of the present invention advantageously provides for an XNA aptamer (e.g., a threomer) that mimics the amino acid residues found at the interface of antibody-antigen interactions. None of the presently known prior references or work has the unique inventive technical feature of the present invention.

Furthermore, the prior references teach away from the present invention. For example, prior references utilize XNA nucleosides monomers that are pre-made. However, the base-modified XNA nucleosides monomers (e.g., TNA nucleosides monomers) of the present invention require XNA nucleosides monomers (e.g., TNA nucleosides monomers) that have to be prepared in-house because they are not commercially available. This in-house preparation requires about 8 synthetic steps, starting from L-ascorbic acid (vitamin C).

Furthermore, the inventive technical features of the present invention contributed to a surprising result. For example, the impact that the modified sidechain had on the ability for TNA aptamers to bind their protein targets. The base-modified aptamers significantly outperformed the standard base aptamers in both quantity and quality.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C shows a DNA display approach to evolving functionally enhanced TNA aptamers. FIG. 1A shows a DNA display design showing the genotype-phenotype relationship of encoding double-stranded (ds) DNA molecules covalently linked to their single-stranded (ss) TNA. TNA molecules that bind a desired protein target are recovered and PCR amplified using primers that are complementary to the primer binding sites (PBS) in the encoding dsDNA. The star represents a fluorescent tag utilized to follow the library through the binding and elution steps of the in vitro selection process. FIG. 1B shows a constitutional structure for the linearized backbone of 3′,2′-α-L-threofuranosyl nucleic acid (TNA) with natural and chemically modified bases mimicking the amino acids phenylalanine and tryptophan. FIG. 1C shows a replication cycle. DNA molecules ligated to a self-priming stem-loop structure are extended with TNA, strand displaced by copying a DNA primer annealed to the loop with DNA and amplified by PCR.

FIGS. 2A, 2B, 2C, 2D 2E, and 2F show the selection performance of functionally enhanced libraries. FIG. 2A shows a Cryo-EM structure of the SARS-CoV-2 spike protein trimer (PDB ID: 6VSB). The RBD domain of the subunit is in the ‘up’ position. FIG. 2B shows the unique reads obtained by bioinformatic analysis of the NGS data collected over the course of the selection against S1. FIG. 2C shows the enrichment values obtained for the top 10,000 most abundant members of each library chemotype against S1. FIG. 2D shows a X-ray crystal structure of the TNF trimer (PDB ID: 2TNF). FIG. 2E shows the unique reads over the course of the selection against TNF. FIG. 2F shows the enrichment values obtained for the top 10,000 most abundant members of each library chemotype against TNF. Table 3 provides a detailed breakdown of the values provided in FIGS. 2B, 20, 2E and 2F.

FIGS. 3A, 3B, and 3C show the kinetic analysis of aptamer binding to a target protein. The left panel of FIGS. 3A and 3B shows the data collected from a single-pass BLI screen of highly enriched sequences at a defined S1 protein (FIG. 3A) or a TNFα protein (FIG. 3B) concentration of 100 nM. The sample size (n) included 30 standard, 20 Phe, and 15 Trp sequences (FIG. 3A) or sample size (n) was 19 standard, 27 Phe, and 20 Trp sequences (FIG. 3B). The right panel of FIG. 3A or 3B shows the full kinetic analysis of 24 highest performing TNA aptamers (FIG. 3A) or of 12 high performing TNA aptamers (FIG. 3B) identified in panel a with detailed kinetic values provided in tabulated form (FIG. 3C). Table 4 provides a detailed breakdown of the kinetic and statistical values provided in FIG. 3A and Table 5 provides a detailed breakdown of the kinetic and statistical values provided in FIG. 3B.

FIGS. 4A, 4B, 4C, and 4D shows the background-subtracted sensorgrams of representative high performing TNA aptamers isolated from the unmodified (Std) and modified (Phe and Trp) libraries. Curve fitting was performed with a 1:1 binding model. The average of three replicates yields K_{D}'s of 34∓11 nM and 3.1∓1.0 nM for standard-2 and Trp-1, respectively (FIGS. 4A and 4B Table 6). The average of three replicates yields K_{D}'s of 82∓24 nM and 20∓6.1 nM for standard-2 and Trp-3, respectively (FIGS. 4C and 4D Table 6).

FIGS. 5A and 5B shows the protein binding activity and specificity of the S1 and TNF threomers. FIG. 5A shows the BLI sensorgrams observed for the Trp-1 and Trp-3 aptamers containing and lacking the indole chemotype against the S1 protein of SARS-CoV-2 and TNFα, respectively. FIG. 5B shows the BLI sensorgrams showing the specificity of the Trp-1 S1 SARS-CoV-2 aptamer against the S1 protein of SARS-CoV-1 and the RBD domain of SARS-CoV-2, and separate BLI sensorgrams showing the specificity of the Trp-3 TNFα aptamer against TNFβ.

FIG. 6A shows the chemical structures of TNA aptamers described herein.

FIGS. 6B and 6C shows the chemical structures of the nucleoside monomers described herein. FIG. 6B shows TNA nucleoside triphosphate monomers synthesized and used in the present invention and FIG. 6C shows TNA phosphoramidite monomers synthesized and used in the present invention.

FIG. 7 shows a primer extension assay. A 5′-labeled DNA primer annealed to a DNA template was extended with TNA using Kod-RSGA. Lane 1: primer only. Lane 2: TNA synthesis with natural bases. Lane 3: TNA synthesis with a tNTP mixture containing tUTP^{Phe} in place of tTTP. Lane 4: TNA synthesis with a tNTP mixture containing tUTP^{Trp} in place of tTTP. Color scheme: DNA (black), TNA (indicated with a line above) with natural bases, and TNA with modified bases (bold). Sequences shown in FIG. 7 are as follow: ggataccacc (SEQ ID NO: 54); ggataccaccuagacgacucacaauagaga (SEQ ID NO: 55); and cctatggtggatctgctgagtgttatctct (SEQ ID NO: 56);

FIGS. 8A and 8B shows representative background-subtracted sensorgrams of the highest performing experimentally-derived aptamer, TNA 913 (FIG. 8A) and computationally-derived aptamer, TNA 949 (FIG. 8B). Curve fitting was performed with a 1:1 binding model. Kinetic values for TNA 913 and 949 are provided in Table 7. Indicated K_{D} values represent the average to two independent replicates.

FIG. 9 shows a schematic representation of the chemical synthesis of 5-iodo-1-(2′-O-benzoyl-α-L-threofuranosyl)-uracil.

FIG. 10 shows a schematic representation of the chemical synthesis of C5-benzyl modified tUTP.

FIG. 11 shows a schematic representation of the chemical synthesis of C5-tryptophan modified tUTP.

FIG. 12 shows a schematic representation of the Chemical synthesis of tNTP with natural bases.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting. All embodiments disclosed herein can be combined with other embodiments unless the context clearly dictates otherwise.

### Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting

As used herein, the term “XNA” or “xeno-nucleic acids” may refer to artificial genetic polymers with novel sugar-phosphate backbones that harbor unique physicochemical properties relative to natural deoxyribonucleic acid (DNA) and ribonucleic acid (RNA) (e.g., properties may include nuclease stability, acid stability, increased thermodynamics of hybridization, or a combination thereof).

As used herein, the term “TNA” or “α-L-threofuranosyl nucleic acid” or “threose nucleic acid” refers to an artificial nucleic acid wherein the sugar portion of the nucleic acid is threose.

As used herein, the term “threomers” are backbone- and base-modified aptamers comprising a TNA sugar moiety and at least one base-modified TNA nucleotide monomer.

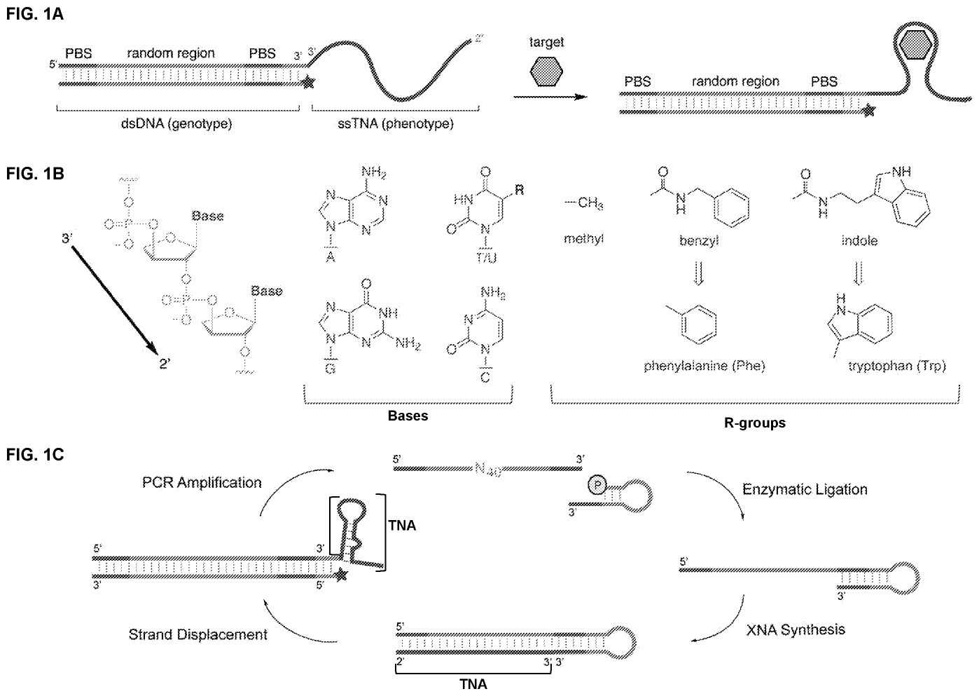

Referring now to FIGS. 1A-12, the present invention features compositions and methods for improving the activity and affinity of in vitro selected XNA aptamers (e.g., TNA aptamers, i.e., threomers) as affinity reagents to protein targets.

The present invention features base-modified xeno nucleic acid (XNA) nucleoside monomers. The base-modified XNA nucleoside monomers may comprise a synthetic, non-natural sugar (e.g., a threose or a hexose sugar), a pyrimidine nucleotide base bound to the sugar moiety (i.e., the synthetic non-natural sugar), and a phosphorus group also bound to the sugar moiety. The pyrimidine nucleotide base may comprise a chemical modification, e.g., at position C-5 of the nucleobase.

Non-limiting examples of xeno nucleic acids (XNA) nucleoside monomers may include but are not limited to threose nucleic acids (TNA) nucleoside monomers, hexose nucleic acids (HNA) nucleoside monomers, locked nucleic acids (LNA) nucleoside monomers, arabino nucleic acid (ANA) nucleoside monomers, fluoroarabino nucleic acid (FANA) nucleoside monomers, or mirror-image DNA or L-DNA nucleoside monomers. In some embodiments, the synthetic, non-natural sugar (i.e., the sugar moiety) is a threose sugar. In other embodiments, the synthetic, non-natural sugar is hexose sugar. In further embodiments, the synthetic, non-natural sugar comprises a modified ribose sugar where the 2′ oxygen and 4′ carbon are connected. In some embodiments, the synthetic, non-natural sugar comprises a ribose sugar or a deoxyribose sugar.

The present invention may also feature a base-modified threose nucleic acid (TNA) nucleoside monomer. The base-modified TNA nucleoside monomer may comprise a threose sugar, a pyrimidine nucleotide base bound to the sugar moiety (i.e., the threose sugar), and a phosphorus group also bound to the sugar moiety. The pyrimidine nucleotide base comprises a chemical modification, e.g., at position C-5 of said pyrimidine nucleotide base.

In some embodiments, the phosphorus group comprises a triphosphate group. In other embodiments, the phosphorus group comprises a phosphoramidite group. The position of where the phosphorus group (e.g., either the triphosphate group or the phosphoramidite group) binds to the sugar moiety of the nucleoside monomers described herein depends on the sugar therein. As used herein, for nucleic acid numbering a prime (′) is used to distinguish atoms on the sugar moiety from those on the nucleobase. The triphosphate group may be bound to the C3′ position on a threose sugar. Or, the triphosphate group may be bound to the C6′ position on a hexose sugar. In some embodiments, the triphosphate group is bound to the C5′ position on a ribose sugar or a deoxyribose sugar. The phosphoramidite group may be bound to the C2′ position on a threose sugar. In an alternative embodiment, the phosphoramidite group may be bound to the C3′ position on a threose sugar. The phosphoramidite group may be bound to the C3′ position on a hexose sugar. In some embodiments, the phosphoramidite group is bound to the C3′ position on a ribose sugar or a deoxyribose sugar.

The nucleotide base of the nucleoside monomers described herein may be bound to the C1′ position of the sugar moiety (e.g., the synthetic, non-natural sugar (e.g., threose or hexose)). In some embodiments, the modified pyrimidine nucleotide base (i.e., the pyrimidine nucleotide base comprising a chemical modification at position C-5 of the nucleobase) is bound to the C1′ position of the sugar moiety. The pyrimidine nucleotide base (e.g., the modified pyrimidine nucleotide base) may comprise a uracil base or a cysteine base. In some embodiments, the pyrimidine nucleotide base may comprise a thymine base. In some embodiments, the pyrimidine nucleotide base (e.g., the modified pyrimidine nucleotide base) may comprise a modified uracil base or a modified cysteine base.

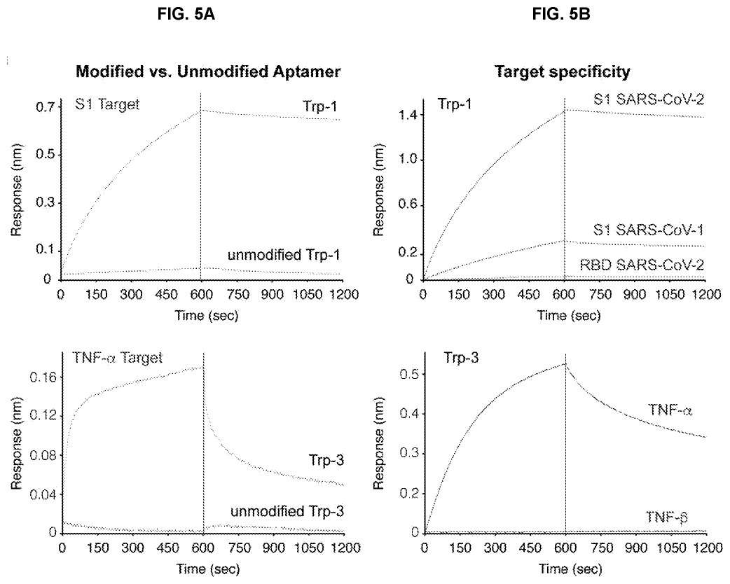

In some embodiments, the pyrimidine nucleotide base described herein comprises a chemical modification at position C-5 of the nucleobase. In some embodiments, the chemical modification at position C-5 of the pyrimidine nucleotide base is a planar aromatic side chain (e.g., a phenylalanine side chain or a tryptophan side chain). Non-limiting examples of the chemical modification include, but are not limited to, phenylalanine side chain, a tryptophan side chain, a leucine side chain, a dioxol side chain, an isopentyl side chain, a dioxethyl side chain, a cyclopropyl side chain, a p-methoxy-phenyl side chain, a napthalene side chain, or a phenethyl side chain (see FIG. 6A or 6B). In some embodiments, the pyrimidine nucleotide base described herein comprises a methyl side chain. Without wishing to limit the present invention to any theories or mechanisms it is believed that a chemical modification comprising a planar aromatic side chain has the ability to mimic amino acid residues that are overrepresented at the paratope-epitope interface of antibody-antigen complexes.

In some embodiments, the present invention may also feature modified purine nucleotide bases. In some embodiments, the purine nucleotide base can be modified at the N-7 position of the ring system. In further embodiments, the chemistry involves synthesizing the purine nucleotide as a 7-deaza-7-modified base.

Additionally, the present invention may feature single-stranded oligonucleotide aptamer (e.g., threomers) for binding a target protein. The aptamers may comprise one or more of the base-modified nucleoside monomers described herein. For example, the present invention may feature a single-stranded threose nucleic acid (TNA) aptamer (i.e., a threomer) for binding a target protein comprising one or more of the base-modified TNA nucleoside monomers as described herein.

The present invention may feature a single-stranded threose nucleic acid (TNA) aptamer (e.g., a threomer) for binding a target protein. The aptamer may comprise one or more of the base-modified TNA nucleotides (i.e., a base-modified TNA nucleoside monomer). The base-modified TNA nucleotide comprising a threose sugar, a pyrimidine nucleotide base comprising a chemical modification at position C-5 bound to the sugar moiety, and a phosphorous group bound to the threose group.

In some embodiments, the aptamers (e.g., threomers) described herein comprise about 20 to 40 nucleoside monomers (e.g., TNA nucleoside monomer) in length. In other embodiments, the aptamers (e.g., threomers) described herein comprise about 15 to 50, or about 15 to 45, or about 15 to 40, or about 15 to 35, or about 15 to 30, or about 15 to 25, or about 15 to 20, or about 20 to 50, or about 20 to 45, or about 20 to 40, or about 20 to 35, or about 20 to 30, or about 20 to 25, or about 25 to 50, or about 25 to 45, or about 25 to 40, or about 25 to 35, or about 25 to 30, or about 30 to 50, or about 30 to 45, or about 30 to 40, or about 30 to 35, or about 35 to 50, or about 35 to 45, or about 35 to 40, or about 40 to 50, or about 40 to 45, or about 45 to 50 nucleoside monomers (e.g., TNA nucleoside monomer) in length. In some embodiments, the aptamers (e.g., threomers) described herein are about 15, or about 20, or about 25, or about 30, or about 35, or about 40, or about 45, or about 50 nucleoside monomers (e.g., TNA nucleoside monomer) in length.

In some embodiments, the aptamers (e.g., threomers) described herein comprise at least one base-modified nucleoside monomer (e.g., a nucleoside monomer with a pyrimidine nucleotide base comprising a chemical modification). In some embodiments, the aptamers comprise at least one modified cysteine monomer. In some embodiments, the aptamers comprise at least one modified uracil monomer. In other embodiments, the aptamers comprise at least one modified cysteine monomer, and at least one modified uracil monomer. In alternative embodiments, the aptamers comprise at least one modified thymine monomer.

In some embodiments, the aptamers (e.g., threomers) described herein is comprised of 25-35% base-modified nucleoside monomer (e.g., a nucleoside monomer with a pyrimidine nucleotide base comprising a chemical modification). In other embodiments, the aptamer is comprised of about 10% to 50%, or about 10% to 45%, or about 10% to 40%, or about 10% to 35%, or about 10% to 30%, or about 10% to 25%, or about 10% to 20%, or about 10% to 15%, or about 15% to 50%, or about 15% to 45%, or about 15% to 40%, or about 15% to 35%, or about 15% to 30%, or about 15% to 25%, or about 15% to 20%, or about 20% to 50%, or about 20% to 45%, or about 20% to 40%, or about 20% to 35%, or about 20% to 30%, or about 20% to 25%, or about 25% to 50%, or about 25% to 45%, or about 25% to 40%, or about 25% to 35%, or about 25% to 30%, or about 30% to 50%, or about 30% to 45%, or about 30% to 40%, or about 30% to 35%, or about 35% to 50%, or about 35% to 45%, or about 35% to 40%, or about 40% to 50%, or about 40% to 45%, or about 45% to 50% base-modified nucleoside monomer (e.g., a nucleoside monomer with a the pyrimidine nucleotide base comprising a chemical modification).

In some embodiments, the nucleoside monomers comprise nucleoside triphosphate monomers. In some embodiments, nucleoside triphosphate monomers comprises base-modified nucleoside triphosphate monomers (e.g., a nucleoside triphosphate monomer with a pyrimidine nucleotide base comprising a chemical modification) (FIG. 6B). In other embodiments, the nucleoside monomers comprise phosphoramidite monomers (i.e., DMT-protected nucleoside phosphoramidate monomers or amidites). In some embodiments, the nucleoside monomers comprise base-modified phosphoramidite monomers (i.e., base-modified DMT-protected nucleoside phosphoramidate monomers or modified amidites) (FIG. 6C).

In some embodiments, the aptamers described herein are for binding a target protein. Non-limiting examples of target proteins include, but are not limited to, a spike protein (S1), a RBD (receptor binding domain) of S1, a tumor necrosis factor (TNF)-α protein, HIV (human immunodeficiency virus) reverse transcriptase, human epidermal growth factor receptor 2 (HER2), trypsin, angiotensin-converting enzyme 2 (ACE2), or thrombin. Other proteins may be targeted in accordance with the composition and methods described herein.

Non-limiting examples of aptamers for binding a spike protein (S1) include, but are not limited to SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20. Non-limiting examples phosphonamidite of aptamers for binding a tumor necrosis factor (TNF)-α protein include but are not limited to SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, or SEQ ID NO: 32.

In some embodiments, the present invention features nucleoside monomers according to the structure structures displayed in Table 1.

TABLE 1
Shows C-5 base-modified nucleoside monomers as described by the present invention.
The R group may be selected from a group consisting of a methyl side chain, a phenylalanine side chain, a
tryptophan side chain, a methyl side chain, a leucine side chain, a dioxol side chain, an isopentyl side chain,
a dioxethyl side chain, a cyclopropyl side chain, a p-methoxy-phenyl side chain, a napthalene side chain,
and a phenethyl side chain (see FIG. 6B or 6C).
Nucleoside Triphosphate Monomers

| | |
|---|---|
| C-5 modified tUTP | C-5 modified tCTP |
| 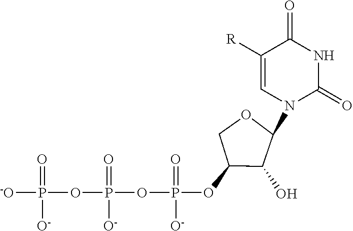 | 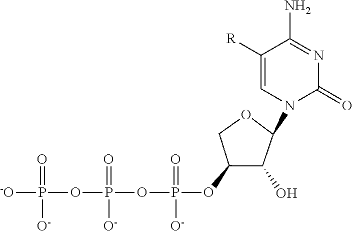 |

Phosphoramidite Monomers

| | |
|---|---|
| C-5 modified tU Amidite | C-5 modified tC Amidite |
| 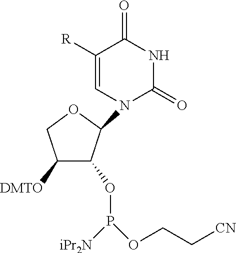 | 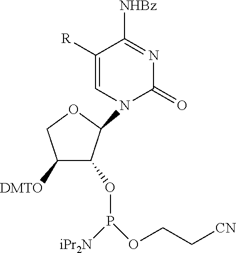 |

The single-stranded threose nucleic acid (TNA) aptamer (e.g., threomer) and/or the single stranded XNA aptamers described herein may be generated through enzymatic and/or chemical synthesis protocols. In some embodiments, the single-stranded threose nucleic acid (TNA) aptamer for binding a target protein comprising one or more base-modified threose nucleic acid (TNA) monomers, is created by using enzymatic synthesis using an engineered TNA polymerase and chemically synthesized TNA triphosphates. In other embodiments, the single-stranded threose nucleic acid (TNA) aptamer for binding a target protein comprising one or more base-modified threose nucleic acid (TNA) monomers, is created by automated solid phase oligonucleotide synthesis using chemically synthesized TNA phosphoramidites.

### Example

The following is a non-limiting example of the present invention. It is to be understood that said example is not intended to limit the present invention in any way. Equivalents or substitutes are within the scope of the present invention.

In an effort to improve the quality of TNA aptamers produced by in vitro selection, the potential for aromatic side chains to enhance the functional properties of large random-sequence TNA libraries was evaluated. The present study focused on tUTP derivatives that are chemically modified at the C5 position with Phe and Trp side chains (FIG. 1B). Importantly, crystal structures of K_{od} DNA polymerase bound to modified DNA provided confidence that the derivatized tUTP nucleotides would be viable substrates for TNA synthesis. The structures reveal a large cavity in the enzyme active site that can accommodate bulky substituents in the major groove of the DNA duplex. A recent crystal structure of Kod-RSGA indicates that the large active site cavity is maintained in the best laboratory evolved TNA polymerase.

First, the desired C5 modified tUTP substrates were synthesized (FIG. 6) following a palladium cross-catalyzed carboxyamidation reaction that couples 5-iodo-1-(2′-O-benzoyl-L-threofuranosyl)-uracil to aromatic amines via in situ formation of an amide linkage. Although other conjugation chemistries are available for linking functional groups to uracil nucleosides, the strategy described herein is a highly versatile route to uniformly modified TNA oligonucleotides. In addition to broad chemical reactivity, the resulting amide linkage (FIG. 1B) benefits from reduced hydrophobicity relative to carbon linkers and limits the number of rotatable bonds between the functional group and nucleobase.

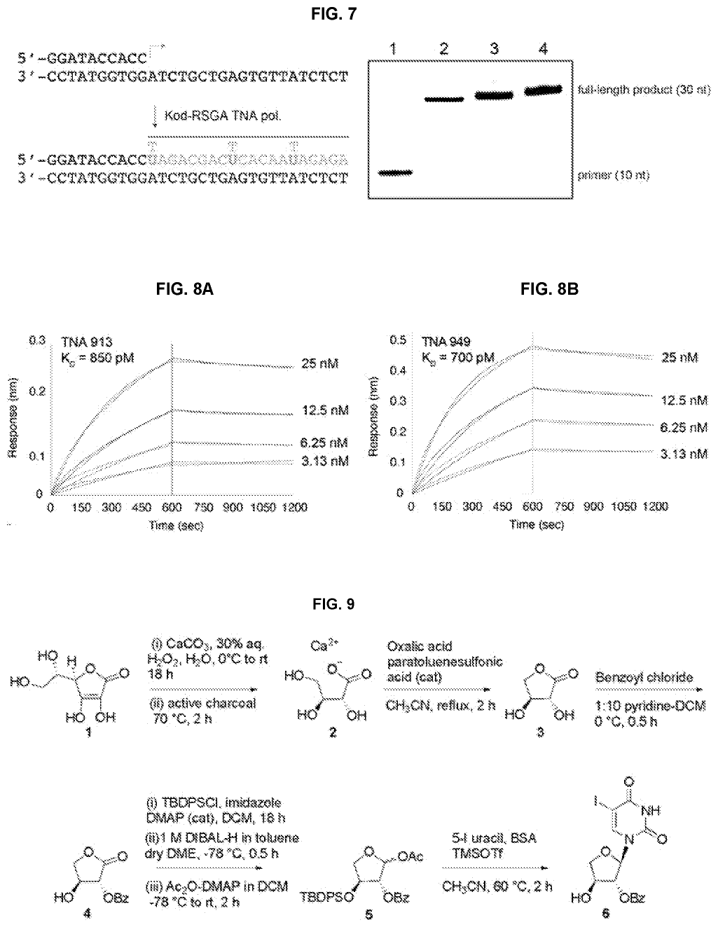

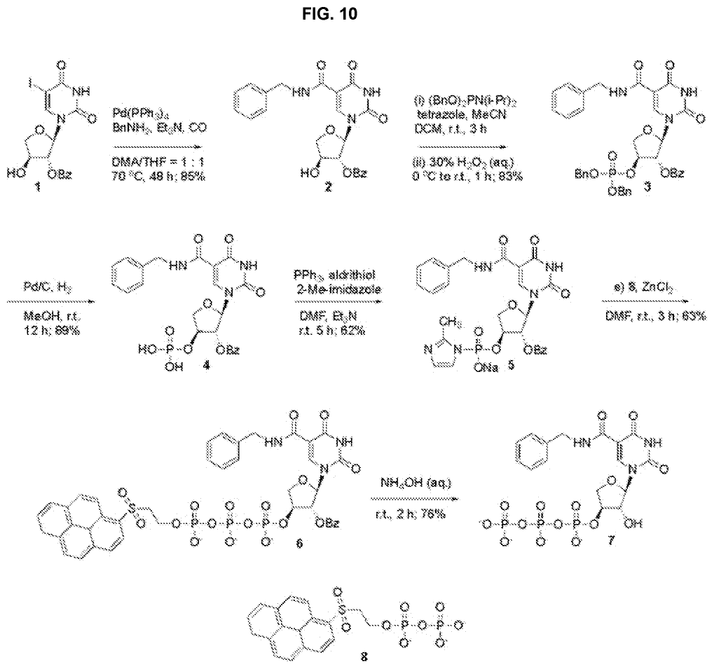

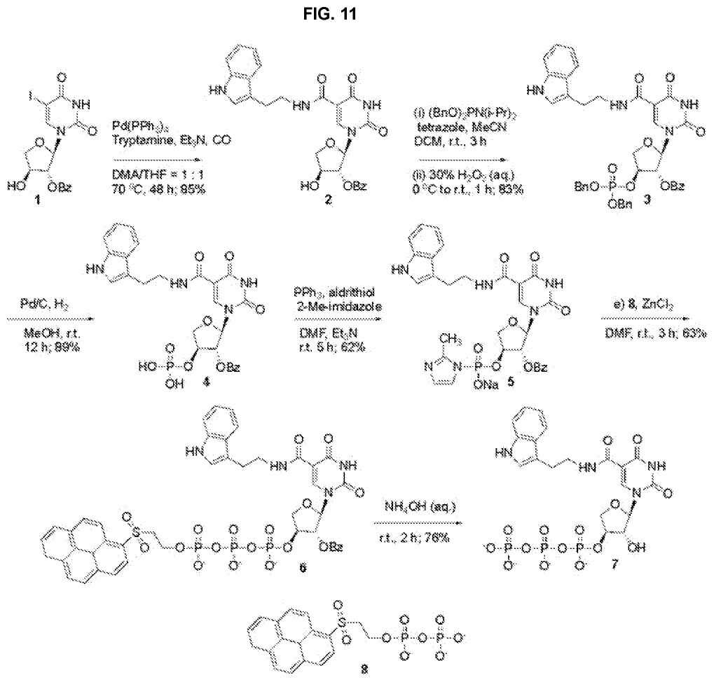

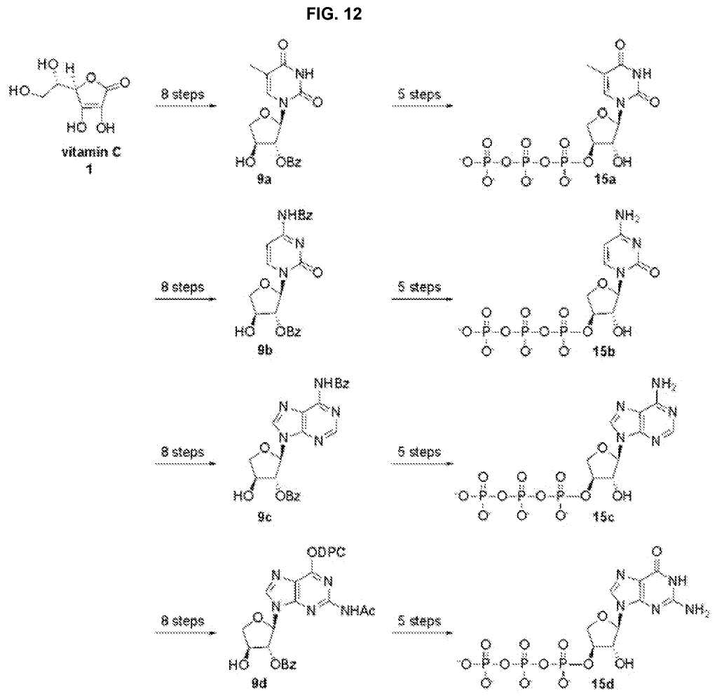

The starting 5-iodo-1-(2′-O-benzoyl-L-threofuranosyl)-uracil nucleoside was prepared in eight steps from L-ascorbic acid (vitamin C) using a Vorbrüggen reaction to conjugate 5-iodo-uracil to an orthogonally protected threose sugar (FIG. 9). Subsequent conjugation of the aromatic side chain to the nucleobase was accomplished in separate reactions by heating either the benzyl or tryptophan amine with the TNA nucleoside in the presence of 10 mol % Pd(Ph₃)₄, and CO (FIGS. 10 and 11). After purification by silica gel chromatography, the C5 modified uracil nucleosides were converted to their corresponding nucleoside-3′-triphosphates by transforming the 3′-hydroxyl group into an activated nucleoside 3′-monophosphate that was coupled to pyrene pyrophosphate to produce a fully protected TNA nucleoside-3′-triphosphate. Following purification, the triphosphate precursor was deprotected in concentrated ammonium hydroxide and precipitated to afford the desired α-L-threofuranosyl uracil nucleoside triphosphates carrying the phenylalanine and tryptophan side chains at the C5 position. This study also required the preparation of all four TNA triphosphates (FIG. 6) with the natural bases of adenine (tATP), cytosine (ICTP), thymine (tTTP), and guanine (tGTP), which were each synthesized in 13 steps from vitamin C (FIG. 12).

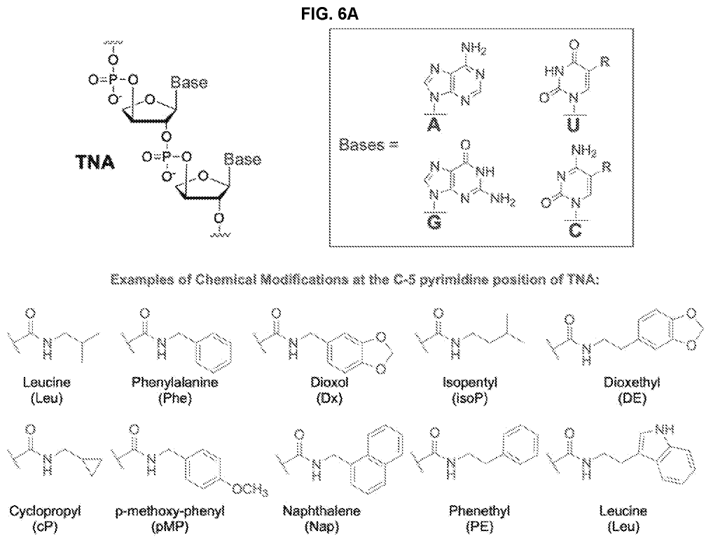

It was determined that the base modified TNA triphosphates were viable substrates for TNA synthesis by evaluating their incorporation into TNA oligonucleotides using a standard primer extension assay. Accordingly, an IR-labeled DNA primer annealed to a DNA template was incubated with a mixture of chemically synthesized tNTPs and Kod-RSGA for 2 hours at 55° C. In these reactions, the tNTP mixture contained either standard bases only or a tNTP solution in which the tTTP substrate was replaced with either tUTP^{Phe} or tUTP^{Trp}. Analysis of the primer extension reactions by denaturing polyacrylamide gel electrophoresis (PAGE) indicates that the primer was extended to full-length product in all cases, as evidenced by the presence of a discrete slower moving band in each lane of the gel (FIG. 7). Further evidence of tUTP^{Phe} and tUTP^{Trp} incorporation into the TNA oligonucleotides was obtained by mass spectrometry (Table 2).

TABLE 2
Mass spectrometry data of TNA oligonucleotides

| SEQ ID | | Theoretical | |
|---|---|---|---|
| NO: | Sequence | mass | Observed mass^{a} |
| 1 | GGATACCACCGAGACGACACACAATAGAGA | 8951.31 | 8958.0 [M + 7H⁺] |
| 2 | GGATACCACCGAGACGACACACAAU^{Phe}AGAGA | 9071.36 | 8984.0 [M − C7H8 + 5H⁺]^{b} |
| 3 | GGATACCACCGAGACGACACACAAU^{Trp}AGAGA | 9123.37 | 9242.0 [M + 5Na⁺ + 4H⁺] |

Underlined: DNA primer region; bold: unmodified base (T) or base modified with benzyl (U^{Phe}) or indole (U^{Trp}) moieties.
^{a}LC-ESI-Q-TOF, ^{b}Partial fragmentation of the benzyl moiety (C₇H₈), similar observation with MALDI-ToF

Encouraged by the synthesis and successful enzymatic incorporation of base-modified tNTPs into TNA, three chemically distinct libraries containing 1014 TNA oligonucleotides were prepared, each displayed on their encoding double-stranded (ds) DNA (FIG. 1A). The libraries were designed at the DNA level to contain a 40 nucleotide (nt) random region that was flanked on the 5′ and 3′ sides with fixed-sequence primer binding sites for PCR amplification. Adjacent to the random region was a 6 nt barcode identifying the library chemotype (standard, Phe, and Trp). The libraries were designed to randomly sample different regions of TNA polymer space for individual sequences that could fold into structures with high affinity to a desired protein target. In particular, the distribution of threomers was evaluated that arise when the same in vitro selection protocol is applied to different TNA library chemotypes. Functionally enhanced libraries should yield a larger abundance of TNA aptamers with higher binding affinity to the desired protein target due to their ability to mimic the paratope of known antibodies.

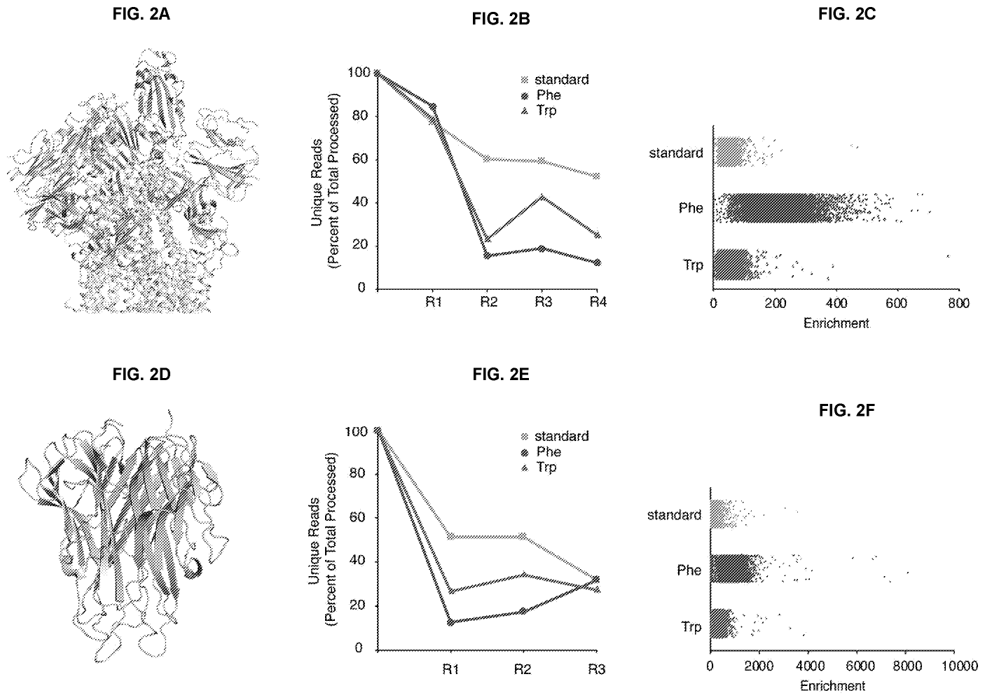

As a model system, TNA sequences were enriched for that bound to the glycosylated form of the S1 subunit of the spike protein of SARS-CoV-2 (FIG. 2A), the causative pathogen of COVID-19. Successive rounds of in vitro selection and amplification (FIG. 1C) were performed in parallel by ligating a DNA stem-loop structure onto a chemically synthesized single-stranded (ss) DNA library. The ligated stem-loop structure serves as the DNA primer for TNA extension across from the DNA template. Kod-RSGA mediated extension of the primer with tNTPs yields a chimeric TNA-DNA heteroduplex, which undergoes strand displacement in a second primer extension step using Bst DNA polymerase to extend a DNA primer annealed to the loop portion of the hairpin with dNTPs. The product of strand displacement is a library of TNA molecules that are each physically linked to their encoding dsDNA templates.

For each round of selection, the TNA libraries were incubated with free Ni-NTA beads to remove any TNA sequences that bound nonspecifically to the solid-support matrix. In round 1, a second negative selection step was performed on the naïve library using His-tagged hemagglutinin (HA), which was chosen as a generic off-target viral-coat protein. The eluted material was incubated with the S1 protein containing a C-terminal His-6 tag. Aptamers that bound specifically to the S1 protein were partitioned away from the unbound material by passing the solution over Ni-NTA beads to capture the aptamer-protein complexes. The beads were washed under high ionic strength conditions to remove weak affinity sequences that bound primarily through electrostatic interactions and the bound material was eluted from the Ni-NTA beads with imidazole. The eluted fractions were exchanged into water and amplified by PCR. Regeneration of the ssDNA library required a second PCR step using a PEG-modified DNA primer that allows for size separation of the PEGylated DNA template by denaturing PAGE. The ssDNA material served as the starting point for the next round of in vitro selection and amplification.

After round 4, the evolving pools of TNA sequences were subjected to high throughput next-generation DNA sequencing (NGS) using an Illumina NovaSeq platform to sequence the elution fractions from each round of selection (Table 3). The combined power of DNA display and NGS bypasses the need for extensive rounds of selection by identifying sequences with enrichment profiles that are indicative of high affinity binders. Bioinformatic analysis of the data reveals that populations containing the Phe and Trp side chains converged more rapidly toward a smaller number of unique sequences as compared to the unmodified library composed entirely of standard bases (FIG. 2B). Enrichment calculations performed on the top 10,000 most abundant sequences observed after four rounds of protein-binding selection indicate a strong enrichment in TNA sequences with Phe side chains. By comparison, TNA sequences equipped with the Trp side chain show only a modest improvement over the unmodified library. However, it's interesting to note that the most enriched sequence derives from the Trp chemotype, suggesting that the Trp population may nevertheless contain several high affinity binders even though the pool exhibited a lower than expected enrichment profile.

TABLE 3
Sequencing reads obtained by NovaSeq.

| | | | | Unique |
|---|---|---|---|---|
| Protein | Base | Round | Processed Reads | Sequences |
| S1 | Standard | 1 | 26,286,663 | 20,412,876 |
| | | 2 | 102,327,271 | 61,923,742 |
| | | 3 | 71,836,059 | 42,735,631 |
| | | 4 | 67,706,251 | 35,428,544 |
| | | Total | 268,156,244 | 160,500,793 |
| | Phe | 1 | 23,583,405 | 20,059,840 |
| | | 2 | 73,162,007 | 11,267,290 |
| | | 3 | 73,322,141 | 13,938,779 |
| | | 4 | 40,614,749 | 4,959,383 |
| | | Total | 210,682,302 | 50,225,292 |
| | Trp | 1 | 30,106,901 | 23,793,731 |
| | | 2 | 94,452,041 | 22,026,578 |
| | | 3 | 68,928,155 | 29,680,238 |
| | | 4 | 71,299,717 | 17,979,790 |
| | | Total | 264,786,814 | 93,480,337 |
| TNFα | Standard | 1 | 518,460,356 | 266,703,781 |
| | | 2 | 367,601,928 | 189,040,364 |
| | | 3 | 591,683,108 | 188,726,113 |
| | | Total | 1,477,745,392 | 644,470,258 |
| | Phe | 1 | 505,151,129 | 63,166,002 |
| | | 2 | 483,294,442 | 84,340,855 |
| | | 3 | 69,009,793 | 21,768,993 |
| | | Total | 1,057,455,364 | 169,275,850 |
| | Trp | 1 | 369,586,308 | 98,436,052 |
| | | 2 | 129,108,753 | 44,024,662 |
| | | 3 | 217,754,947 | 59,876,218 |
| | | Total | 716,450,008 | 202,336,932 |

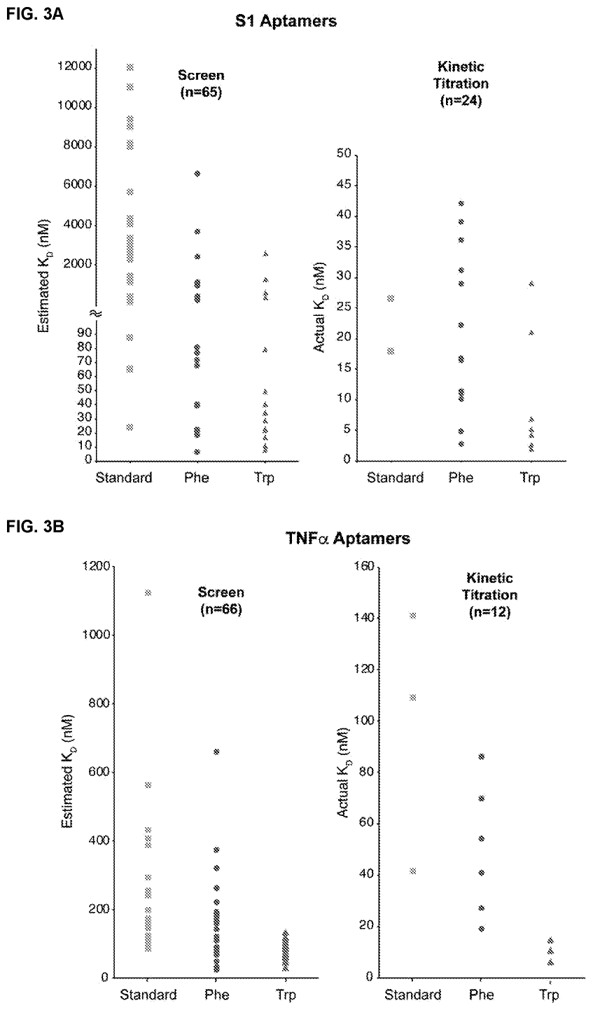

To better illuminate the relationship between sequence enrichment and function, members of each chemotype family were screened for affinity to the S1 protein. In this experiment, 65 sequences with promising enrichment profiles were selected and individually synthesized by polymerase-mediated primer extension on complementary DNA templates. Estimated binding affinities (K_{D}) were determined by biolayer interferometry (BLI) using biotinylated aptamers that were immobilized onto the surface of streptavidin-coated biosensors and assayed for binding activity to S1 poised at a protein concentration of 100 nM. The screen revealed striking differences in the number of high-affinity aptamers produced from the modified libraries versus those obtained from the standard base library. A plot of estimated K_{D} value versus library chemotype highlights these differences, which bin according to 3380 nM (n=30), 910 nM (n=20), and 430 nM (n=15) for the average binding affinities observed for the standard, Phe, and Trp libraries, respectively (FIG. 3A). Analysis of the data indicates that more than half of the functionally enhanced aptamers are predicted to bind the S1 protein with a K_{D} of <90 nM versus only 10% for the standard base library. In addition to reduced binding affinity, the standard library shows a wider range of K_{D} values, indicating the pool either contains fewer high affinity binders or is less convergent, which is consistent with the higher number of unique reads observed in the NGS data (FIG. 2B). Together, these data support the hypothesis that expanded chemical functionality is necessary for generating threomers with higher protein binding affinity.

To more precisely evaluate the binding properties of the in vitro selected TNA aptamers isolated from the three chemotype libraries, full kinetic measurements were performed on a subset of the sequences that were predicted to bind S1 with a K_{D} value of <50 nM. In total, 24 TNA aptamers were re-synthesized for further characterization by BLI (FIG. 3B, Table 4). Curve fitting reveals that all of the data conform to a 1:1 binding model with the top 8 binders having K_{D} value in the range of 1 nM-10 nM, and deriving exclusively from the functionally enhanced libraries. In general, TNA aptamers isolated from functionally enhanced libraries bind S1 with K_{D} values that are >10-fold lower than aptamers isolated from the standard library. Moreover, modified aptamers exhibit higher binding responses (>2-fold) to the aptamer-coated biosensor than unmodified aptamers, indicating a stronger aptitude for overall binding to the S1 protein.

TABLE 4
BLI analysis of S1 aptamers chosen for full kinetics.

| | | | | | | | |
|---|---|---|---|---|---|---|---|
| SEQ ID | | TNA Sequence | K_{D} | kₒₙ | k_{off} | Fit | Variance |
| NO: | Entry | (3′ to 2′) | (nM) | (M⁻¹s⁻¹) | (s⁻¹) | (R²) | (Chi²) |

Standard

| | | | | | | | |
|---|---|---|---|---|---|---|---|
| 4 | 1 | TGGCGAGGGGTAGTGAGCTG | 17.6 | 1.6 × | 2.7 × | 0.99 | 0.20 |
| | | ACGGATAGGGCTAGTGGAAC | | 10⁴ | 10⁻⁴ | | |
| 5 | 2 | CGTGCACGATCTAGCCAGT | 27.3 | 9.0 × | 2.5 × | 0.99 | 0.16 |
| | | GCTCGCTTGAAATGGGCGC | | 10³ | 10⁻⁴ | | |
| | | AG | | | | | |

Phe

| | | | | | | | |
|---|---|---|---|---|---|---|---|
| 6 | 3 | TATTGGAACTCCCGAGCAGG | 2.6 | 9.0 × | 2.4 × | 0.99 | 0.43 |
| | | CTGTCCCATGGAAGACTACA | | 10⁴ | 10⁻⁴ | | |
| 7 | 4 | AGGCTTATGAAATCTTATGT | 5.2 | 1.4 × | 7.5 × | 0.99 | 2.69 |
| | | GAACCGGGCATACGGGCGA | | 10⁴ | 10⁻⁵ | | |
| 7 | 5* | AGGCTTATGAAATCTTATGT | 10.0 | 1.8 × | 1.8 × | 0.99 | 0.41 |
| | | GAACCGGGCATACGGGCGA | | 10⁴ | 10⁻⁴ | | |
| | | T | | | | | |
| 8 | 6 | GAGAGTCATAGTAGTTCACG | 11.3 | 1.5 × | 1.7 × | 0.99 | 0.70 |
| | | GGTCTGCAGCTTACGCATGT | | 10⁴ | 10⁻⁴ | | |
| 8 | 7* | GAGAGTCATAGTAGTTCACG | 12.0 | 2.5 × | 3.0 × | 0.99 | 0.61 |
| | | GGTCTGCAGCTTACGCATGT | | 10⁴ | 10⁻⁴ | | |
| 6 | 8* | TATTGGAACTCCCGAGCAGG | 16.3 | 1.2 × | 2.0 × | 0.99 | 0.22 |
| | | CTGTCCCATGGAAGACTACA | | 10⁴ | 10⁻⁴ | | |
| 9 | 9 | TGTACGCCATATAGGCAGTC | 16.7 | 1.2 × | 1.9 × | 0.99 | 0.29 |
| | | ATCGAGCAACGAAGTAATGG | | 10⁴ | 10⁻⁴ | | |
| 7 | 10* | AGGCTTATGAAATCTTATGT | 21.8 | 1.2 × | 2.7 × | 0.99 | 4.30 |
| | | GAACCGGGCATACGGGCGA | | 10⁴ | 10⁻⁴ | | |
| | | T | | | | | |
| 10 | 11 | ACGATGGACTTGGGTAGCTA | 22.1 | 9.5 × | 2.1 × | 0.99 | 0.73 |
| | | GTCAGGTTAGGCTGTTTGGA | | 10³ | 10⁻⁴ | | |
| 11 | 12 | AGAACTTCGTCAGCGTCTTA | 28.8 | 9.1 × | 2.6 × | 0.99 | 0.71 |
| | | CCGAGTCCAAGGACTGGCT | | 10³ | 10⁻⁴ | | |
| | | A | | | | | |
| 12 | 13 | ATCGAGTTGTGACGTTAACG | 31.0 | 2.0 × | 6.2 × | 0.99 | 0.14 |
| | | ATAATAGCCCGCTCCGATTC | | 10⁴ | 10⁻⁴ | | |
| 13 | 14 | TACATTACGCGTTAGCGTTA | 36.0 | 9.4 × | 3.3 × | 0.99 | 26.65 |
| | | ATGGGTATGTCCGGACAGT | | 10³ | 10⁻⁴ | | |
| | | G | | | | | |
| 14 | 15 | GGTGTAATAAGGAGAGTACG | 39.0 | 4.3 × | 1.7 × | 0.99 | 40.40 |
| | | TCGTTTGGTGGGACCGGGT | | 10³ | 10⁻⁴ | | |
| | | A | | | | | |
| 13 | 16* | TACATTACGCGTTAGCGTTA | 42.0 | 5.2 × | 2.2 × | 0.99 | 3.30 |
| | | ATGGGTATGTCCGGACAGT | | 10³ | 10⁻⁴ | | |
| | | G | | | | | |

Trp

| | | | | | | | |
|---|---|---|---|---|---|---|---|
| 15 | 17 | TTGGCGAGATCCCCTATACG | 2.0 | 6.7 × | 1.3 × | 0.99 | 2.94 |
| | | TGAGCTTCCTTTTATCCTTT | | 10⁴ | 10⁻⁴ | | |
| 16 | 18 | CGTACAACTCCGGTTTCGGG | 2.7 | 5.2 × | 1.4 × | 0.99 | 0.52 |
| | | CGGCCCGTTCTTCAGTTTGA | | 10⁴ | 10⁻⁴ | | |
| 17 | 19 | TAAGGAAAGGGGAGCGGGT | 4.2 | 3.3 × | 1.4 × | 0.99 | 0.73 |
| | | AGGAGGTTCGTCGGGTGCG | | 10⁴ | 10⁻⁴ | | |
| | | AC | | | | | |
| 18 | 20 | ATTGTGCGAAGTATACATAG | 5.1 | 2.1 × | 1.1 × | 0.99 | 0.12 |
| | | CAGATGCGTGACATAACAAG | | 10⁴ | 10⁻⁴ | | |
| 16 | 21* | CGTACAACTCCGGTTTCGGG | 6.9 | 9.7 × | 6.7 × | 0.99 | 2.02 |
| | | CGGCCCGTTCTTCAGTTTGA | | 10⁴ | 10⁻⁴ | | |
| 19 | 22 | AAACTGTTGGGACTTGTAAT | 21.1 | 8.7 × | 1.8 × | 0.72 | 16.72 |
| | | GTCGTCATCAGCTAAGAGTC | | 10⁴ | 10⁻³ | | |
| 20 | 23 | TTTCTATGCATCTCGGGGAT | 29.1 | 3.9 × | 1.1 × | 0.97 | 1.85 |
| | | GCGGGATATAAAAGAGAGA | | 104 | 10⁻³ | | |
| | | C | | | | | |
| 20 | 24* | TTTCTATGCATCTCGGGGAT | 76.0 | 7.0 × | 5.4 × | 0.98 | 30.24 |
| | | GCGGGATATAAAAGAGAGA | | 10³ | 10⁻⁴ | | |
| | | C | | | | | |

*Signifies an independent replicate, PBS8 primer sequence not shown (SEQ ID NO: 37)

TABLE 5
BLI analysis of TNFα aptamers chosen for full kinetics.
OTHER indicates a base modified with benzyl moeity
alpha-L-threofuranosyl nucleic acid

| | | | | | | | |
|---|---|---|---|---|---|---|---|
| SEQ | | | | | | | |
| ID | | | K_{D} | kₒₙ | k_{off} | Fit | Variance |
| NO: | Entry | TNA Sequence (3′ to 2′) | (nM) | (M⁻¹s⁻¹) | (s⁻¹) | (R²) | (Chi²) |

Standard

| | | | | | | | |
|---|---|---|---|---|---|---|---|
| 21 | 1 | GCGGGGGGTGTTGTGTAACTGTT | 42 | 1.1 × 10⁵ | 4.4 × | 0.88 | 0.20 |
| | | TTTTGCCGGAAAGACGC | | | 10⁻³ | | |
| 22 | 2 | GACGCTATGACTGGTATGGTGTG | 109 | 1.2 × 10⁵ | 1.3 × | 0.92 | 0.15 |
| | | TGAGCTTCGAGGTCTGA | | | 10⁻² | | |
| 23 | 3 | CGCGGCGTTGCGTAGCGCCAAT | 141 | 7.5 × 10⁴ | 1.1 × | 0.88 | 0.11 |
| | | CCTGCACAGAGGCGGGCG | | | 10⁻² | | |

Phe

| | | | | | | | |
|---|---|---|---|---|---|---|---|
| 24 | 4 | TGACACCTGGCACGAATGCGCA | 19 | 2.6 × 10⁵ | 5.0 × | 0.89 | 0.07 |
| | | GACGACGTTGTTTTGCGT | | | 10⁻³ | | |
| 25 | 5 | ACTTTTGCTTGAGATTCGGCGCA | 27 | 2.9 × 10⁵ | 7.8 × | 0.91 | 0.15 |
| | | TTTCGTGCAGTGATGTA | | | 10⁻³ | | |
| 26 | 6 | CTTAGGTCGGCCTTTGACCATTC | 41 | 3.0 × 10⁵ | 1.2 × | 0.94 | 0.18 |
| | | GTGCAAAACTTAGCGAA | | | 10⁻² | | |
| 27 | 7 | GAATGCGCTTGAGCGCGCCCGA | 54 | 1.7 × 10⁵ | 9.1 × | 0.87 | 0.11 |
| | | TGATATAGCGAACCAAGC | | | 10⁻³ | | |
| 28 | 8 | TTAATTTTGAGATCTTTGTCGAGT | 70 | 2.2 × 10⁵ | 1.5 × | 0.98 | 0.24 |
| | | TGCTCGGTGAAAAGTC | | | 10⁻² | | |
| 29 | 9 | AGAGCAAGTCATGATCGCGAGTT | 86 | 2.3 × 10⁵ | 2.0 × | 0.97 | 0.14 |
| | | TAGCTTTTTCATTTCCC | | | 10⁻² | | |

Trp

| | | | | | | | |
|---|---|---|---|---|---|---|---|
| 30 | 10 | GGCACTATCGATTATCGTGCGCA | 7.3 | 9.8 × 105 | 7.1 × | 0.97 | 0.07 |
| | | TTGGTTGTGTCCAGGCT | | | 10⁻³ | | |
| 31 | 11 | TCACGTCTGTTGTACACTTGTGC | 12 | 4.6 × 105 | 5.4 × | 0.99 | 0.17 |
| | | AATGCAAGGACGGAAAA | | | 10⁻³ | | |
| 32 | 12 | ACGAGTAGGCCGCATTATGATGA | 16 | 3.3 × 105 | 5.2 × | 0.99 | 0.37 |
| | | TTTAAACTGTTTCATGC | | | 10⁻³ | | |

PBS8 primer sequence not shown (SEQ ID NO: 37)

To confirm the reproducibility of the data, a representative high performance S1 aptamer was evaluated from the standard and modified chemotype libraries in triplicate using independently synthesized material for each kinetic binding assay. The best modified and unmodified S1 aptamers (designated Trp-1 and standard-2, respectively) have average affinity values of 3.1∓1.0 nM and 34∓11 nM, respectively, for the S1 protein of SARS-CoV-2 (FIG. 3C, Table 6). These values, which closely approximate the K_{D} values observed in the kinetic titration assay, provide confidence that the data provided in FIG. 3B is an accurate reflection of the binding activity of the evolved threomers. Analysis of the BLI sensorgrams indicates the enhanced activity of Trp-1 is due to a combination of faster on-rates, slower-off rates, and higher overall binding to the aptamer-coated biosensor than the unmodified standard-2 aptamer.

TABLE 6
BLI analysis of high performing standard and modified S1 and TNFα aptamers chosen
for replicative trials.

| | | | | | | | |
|---|---|---|---|---|---|---|---|
| SEQ ID | | | K_{D} | kₒₙ | k_{off} | Fit | Variance |
| NO: | Aptamer | TNA Sequence (3′-2′) | (nM) | (M⁻¹s⁻¹) | (s⁻¹) | (R²) | (Chi²) |

S1 protein Target

| 5 | Standard-2 | CGTGCACGATCTAGCC | 27 | 9.0 × | 2.5 × | 0.99 | 0.16 |
|---|---|---|---|---|---|---|---|
| | | AGTGCTCGCTTGAAAT | | 10³ | 10⁻⁴ | | |
| | | GGGCGCAG | 46 | 5.5 × | 2.6 × | 0.99 | 0.58 |
| | | | | 10³ | 10⁻⁴ | | |
| | | | 28 | 9.8 × | 2.7 × | 0.99 | 0.74 |
| | | | | 10³ | 10⁻⁴ | | |
| | | avg ± std | 34 ± 11 | | | | |
| 15 | Trp-1 | TTGGCGAGATCCCCTA | 2.0 | 6.7 × | 1.3 × | 0.99 | 2.94 |
| | | | | 10⁴ | 10⁻⁴ | | |
| | | TACGTGAGCTTCCTTTT | 3.8 | 3.6 × | 1.4 × | 0.99 | 0.23 |
| | | | | 10⁴ | 10⁻⁴ | | |
| | | ATCCTTT | 3.5 | 3.6 × | 1.3 × | 0.99 | 0.66 |
| | | | | 10⁴ | 10⁻⁴ | | |

| | |
|---|---|
| avg ± std | 3.1 ± 1.0 |

TNFα

| 22 | Standard-2 | GACGCTATGACTGGTA | 109 | 1.2 × | 1.3 × | 0.92 | 0.15 |
|---|---|---|---|---|---|---|---|
| | | TGGTGTGTGAGCTTCG | | 10⁵ | 10⁻² | | |
| | | AGGTCTGA | 76 | 7.5 × | 5.7 × | 0.94 | 0.28 |
| | | | | 10⁴ | 10⁻³ | | |
| | | | 62 | 9.2 × | 5.7 × | 0.95 | 0.20 |
| | | | | 10⁴ | 10⁻³ | | |
| | | avg ± std | 82 ± 24 | | | | |
| 32 | Trp-3 | ACGAGTAGGCCGCATT | 16 | 3.3 × | 5.2 × | 0.99 | 0.37 |
| | | ATGATGATTTAAACTGT | | 10⁵ | 10⁻³ | | |
| | | TTCATGC | 17 | 5.8 × | 9.9 × | 0.99 | 0.09 |
| | | | | 10⁵ | 10⁻³ | | |
| | | | 27 | 2.6 × | 7.1 × | 0.99 | 0.23 |
| | | | | 10⁵ | 10⁻³ | | |

| | |
|---|---|
| avg ± std | 20 ± 6.1 |

avg, average; std, standard deviation
Each data set represents an independent replicate with freshly synthesized aptamer
PBS8 primer sequence not shown (SEQ ID NO: 3)

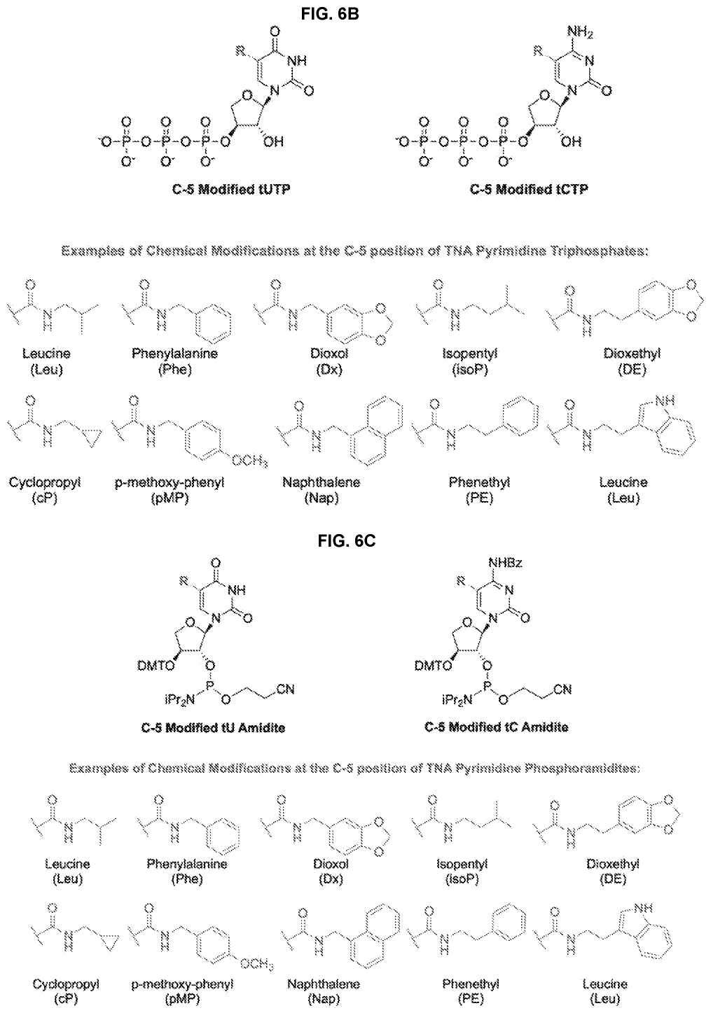

Next, the binding affinity of threomers were improved by comparing experimental and computational methods for directed evolution. A doped library based on the Trp-1 sequence was synthesized that contained 85% identity and 15% diversity at each nucleotide position with a theoretical diversity of ˜3×10⁹ unique members. The library was subjected to 1 round of selection for binding to S1 followed by NGS analysis to identify individual sequences present in the eluted material. The best experimental aptamer identified from ˜30 candidate sequences identified through a combination of enrichment and sequence context exhibited a K_{D} of 850∓160 μM for S1 (FIGS. 8A and 8B, Table 7). By comparison, a parallel machine learning-based approach, inspired by recent neural network approaches in sequence design that rely on sequence and functional information obtained from the original S1 selection yielded a threomer with a K_{D} of 700∓40 μM from a screen of ˜12 candidate sequences (FIGS. 8A and 8B, Table 7). This latter observation suggests that in silico methods may offer a faster round to sequence optimization than traditional directed evolution.

TABLE 7
BLI analysis of directed evolution aptamers by experimental and computational
approaches:

| | | | | | | | |
|---|---|---|---|---|---|---|---|
| SEQ ID | | | K_{D} | kₒₙ | k_{off} | Fit | Variance |
| NO: | Aptamer | TNA Sequence (3′-2′) | (nM) | (M⁻¹s⁻¹) | (s⁻¹) | (R²) | (Chi²) |

S1 protein

| 33 | TNA 913 | GCGGCCAGATCCCCGA | 0.96 | 1.2 × | 1.2 × | 0.99 | 0.53 |
|---|---|---|---|---|---|---|---|
| | (experimental) | TATGTTAACGTCCTTTT | | 10⁵ | 10⁻⁴ | | |
| | | ATGCGGG | 0.73 | 1.2 × | 8.9 × | 0.99 | 0.42 |
| | | | | 10⁵ | 10⁻⁵ | | |
| | | avg ± std | 0.85 ± 0.16 | | | | |
| 34 | TNA 949 | TTAGTATTATTATGGGT | 0.67 | 1.3 × | 8.8 × | 0.99 | 0.96 |
| | (computational) | GCGTGAGTTTCCAAGA | | 10⁵ | 10⁻⁵ | | |
| | | AGCGCGT | 0.73 | 1.7 × | 1.2 × | 0.99 | 1.7 |
| | | | | 10⁵ | 10⁻⁴ | | |

| | |
|---|---|
| avg ± std | 0.7 + 0.04 |

avg, average; std, standard deviation
Each data set represents an independent replicate with freshly synthesized aptamer
PBS8 primer sequence not shown (SEQ ID NO: 37)
Kinetic plots provided in FIG. 8A and 8B.

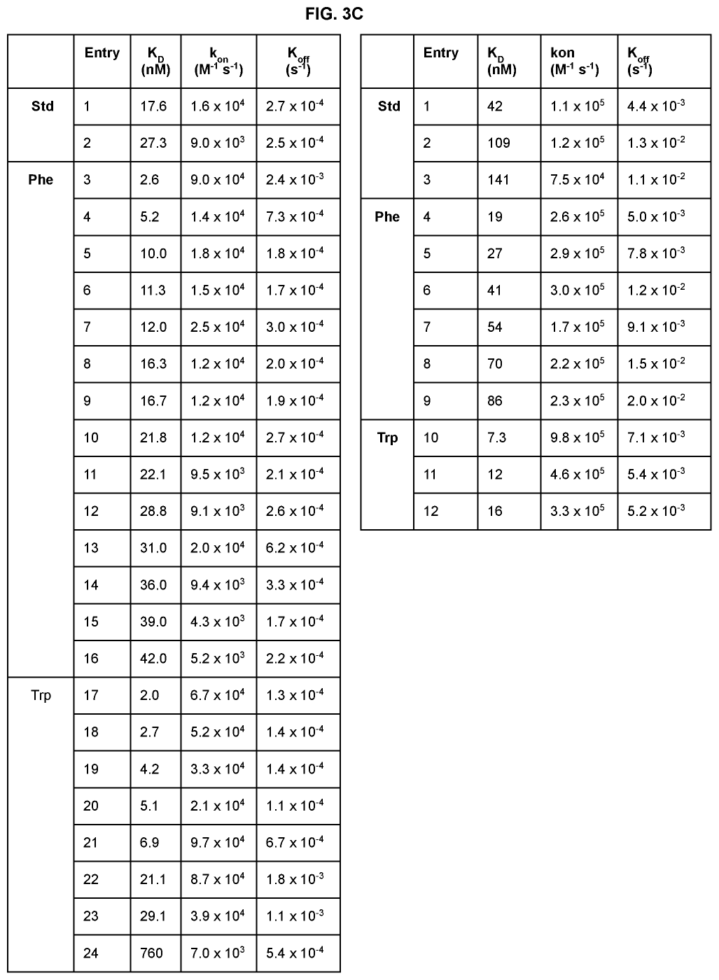

To evaluate the generality of functionally enhanced TNA libraries as a rich source of chemical diversity for producing superior TNA-based affinity reagents, a similar in vitro selection experiment was performed against a different protein target. For this study, tumor necrosis factor-alpha (TNF, FIG. 2D), a cytokine that is closely linked to inflammatory diseases was chosen. NGS analysis of the elution fractions obtained after 3 rounds of in vitro selection and amplification for TNA aptamers with affinity to TNF indicates that TNA libraries carrying the Phe and Trp chemotypes exhibit higher levels of enrichment than the standard base library (FIGS. 2E and 2F). An activity screen of 66 TNA aptamers reveals an estimated K_{D} range of 24-1123 nM with most of the Phe- and Trp-modified aptamers having K_{D} values that cluster below 200 nM (FIG. 4A). A full kinetic analysis of the top performing aptamers from each chemotype library uncovered a strong preference for Trp-modified aptamers (FIG. 3B). A comparison of representative high performing standard and modified TNA aptamers (FIG. 3C) reveals that the higher activity of the modified aptamers is primarily due to slower off-rates in the protein-aptamer binding equilibrium. In this case, the best aptamers isolated from the standard and modified chemotype libraries (designated standard-2 and Trp-3, respectively) bind with average K_{D} values of 82∓24 nM and 20∓76.1 nM (FIG. 4C, Table 6), respectively, when assayed as three independent replicates. However, the K_{D} observed for the best standard base aptamer is only an approximate value due to poor curve fitting caused by weak binding. As with the S1 aptamers, modified aptamers immobilized on the streptavidin-coated biosensor exhibit higher binding responses than the unmodified aptamers. These data further support the general conclusion that functionally enhanced TNA libraries improve the quantity and quality of high activity threomers generated by in vitro selection.

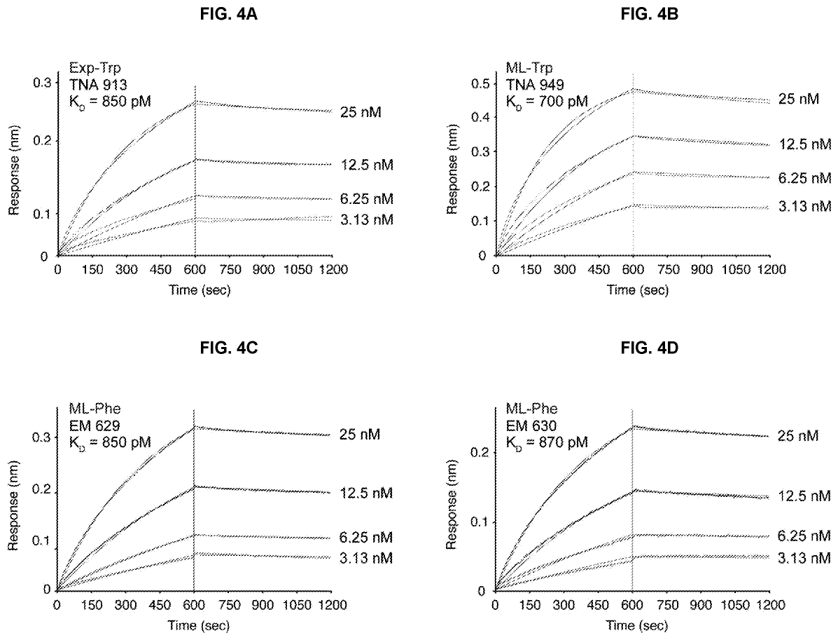

Last, the contribution of the modified side chains toward the activity and specificity of threomer binding to the S1 and TNF-α target proteins was evaluated. The importance of the hydrophobic amino acid side chains was evaluated by measuring the binding affinity of modified and unmodified versions of the Trp-1 and Trp-3 aptamers selected to bind S1 and TNF-α, respectively. In both cases, the resulting BLI sensorgrams clearly show that protein binding affinity is completely abrogated when the in vitro selected TNA sequences are synthesized with the natural base chemotype (FIG. 5A). To investigate the potential for protein target binding specificity, the Trp-1 and Trp-3 aptamers were challenged to bind closely related protein targets. The resulting BLI sensorgram reveals that Trp-1 exhibits a strong preference for the S1 protein of SARS-CoV-2 versus SARS-CoV-1 (FIG. 5B). An even more striking result was observed for the Trp-3 threomer, which shows no detectable binding for TNF-β (FIG. 5D). Interestingly, the BLI analysis also reveals that Trp-1 binds outside the RBD domain of the S1 protein, suggesting that additional screening or selections could lead to an RBD binder that could be used in a sandwich assay or as a therapeutic candidate.

The data collected in the present study provides compelling evidence that functionally enhanced TNA libraries produce higher affinity binders than standard TNA libraries carrying only natural bases. This result was achieved through a combination of DNA display and high throughput NGS sequencing, which allowed for the isolation of base-modified threomers with K_{D} values in the low nM range after only 3-4 rounds of selective amplification. The absence of significant numbers of high affinity binders from the unmodified library supports the hypothesis that planar aromatic side chains have the ability to mimic amino acid residues that are overrepresented at the paratope-epitope interface of antibody-antigen complexes. The generality of the conjugation chemistry established to construct uniformly modified TNA libraries is sufficiently versatile that it should be possible to explore new regions of chemical space by synthesizing a broader range of C5 modified tUTP substrates.

The path to functionally enhanced XNA libraries was limited by a number of synthetic challenges that are less severe for DNA-based affinity reagent technologies. TNA, like most XNAs, is assembled from building blocks that are not commercially available, and thus, must be obtained by chemical synthesis. In this case, a complete monomer set of all four TNA nucleoside triphosphates (tNTPs) requires 52 chemical transformations to convert vitamin C into each of the four tNTPs (tATP, tCTP, tTTP, and tGTP). The tUTP^{Phe} and tUTP^{Trp} substrates demanded an additional 30 chemical transformations (15 steps each) with the critical step being the palladium-catalyzed cross-coupling reaction required to functionalize the uracil nucleobase with an aromatic side chain. Once the substrates are prepared, it then becomes necessary to identify a polymerase that can synthesize functionally enhanced TNA oligonucleotides. In the current study, this was accomplished using Kod-RSGA, a recently evolved TNA polymerase developed for enhanced TNA synthesis activity on DNA templates.

Although antibodies remain the gold standard as protein affinity reagents, aptamers have a number of advantages that have caused them to grow in popularity. In addition to prolonged storage and shipping at ambient temperature, aptamers are produced by cell-free synthesis (chemical or enzymatic), which avoids viral or bacterial contamination problems associated with cellular protein production systems and allows for greater scalability. Aptamers have low immunogenicity, low batch-to-batch variability, and greater ease of chemical modifications than antibodies and other protein-based affinity reagents. However, despite these advantages, aptamers have historically suffered from poor biological stability and fast off-rates. The current study is part of a program of research designed to narrow the gap between antibodies and aptamers. The goal has been to establish an artificial genetic system that is both biologically stable and amenable to Darwinian evolution, and then to augment this system with the chemical functionality required to achieve high affinity interactions that are driven by slow off-rate binding kinetics.

In conclusion, the present invention has established threomers as a new class of biologically stable affinity reagents that function with enhanced target-binding activity.

### General Information

Taq DNA polymerase, Bst 3.0 DNA polymerase, T4 DNA ligase, 10×T4 DNA ligase buffer, and 10× ThermoPol buffer were purchased from New England Biolabs (Ipswich, MA). PCRBIO HiFi polymerase was purchased from PCRBiosystems (Wayne, PA). Kod RSGA TNA polymerase was expressed and purified. Experiments were performed in DNA LoBind tubes, purchased from Eppendorf (Hamburg, Germany). DNA triphosphates were purchased from Thermo Fisher Scientific (Waltham, MA). TNA triphosphates bearing natural bases were synthesized. DNA oligonucleotides were purchased from Integrated DNA Technologies (Coralville, IA), purified by denaturing polyacrylamide gel electrophoresis (PAGE), electroeluted, buffer exchanged and concentrated using Millipore YM-3 or YM-30 Centricon centrifugal filter units, and quantified by UV absorbance via NanoDrop (Thermo Fisher Scientific). For the aptamer selection, Ni-NTA agarose resin was purchased from Qiagen (Hilden, Germany). His-tagged SARS-CoV-2 spike glycoprotein subunit 1 (S1), SARS-CoV-2 receptor binding domain (RBD) of spike glycoprotein subunit 1, and SARS-CoV-1 spike glycoprotein subunit 1 (S1) were purchased from ACROBiosystems (Newark, DE). His-tagged TNFα was expressed from E. coli as previously described. His-tagged TNFβ was purchased from Sino Biological (Wayne, PA). Streptavidin and Ni-NTA biosensor were purchased from ForteBio (Fremont, CA).

TABLE 8
DNA oligonucleotides used in the study

| | | Sequence (5′→3′) Modifications in IDT nomenclature. N represents |
|---|---|---|
| SEQ ID | | a 30 A:20 C:20 G:30 T combination. N′ represents a combination of |
| NO: | Name | 85 parent nucleobase:15 other three nucleobases (5 each). |
| 35 | PBS7 | GGATCGTCAGTGCATTGAGA |
| 36 | PEG PBS7 | AACAAACAAACAAACAAACAAACAAACAAACA/iSp9/GGATCGTCAGTG |
| | | CATTGAGA |
| 37 | PBS8 | GTCCCCTTGGGGATACCACC |
| 38 | PBS12 | TTCTCATGTTTGACAGCTTA |
| 39 | PEG PBS12 | AACAAACAAACAAACAAACAAACAAACAAACA/iSp9/TTCTCATGTTTGA |
| | | CAGCTTA |
| 40 | PBS13 | TCATCGATAAGCTTTAATGC |
| 41 | PEG PBS13 | AACAAACAAACAAACAAACAAACAAACAAACA/iSp9/TCATCGATAAGC |
| | | TTTAATGC |
| 42 | PBS15 | TTGCTAACGCAGTCAGGCAC |
| 43 | PEG PBS15 | AACAAACAAACAAACAAACAAACAAACAAACA/iSp9/TTGCTAACGCAG |
| | | TCAGGCAC |
| 44 | DNA Display | /5Phos/ACTACGTACCCACAACCTCGGCCGTACCACGGTACGTAGTGT |
| | Hairpin | CCCCTTGGGGATACCACC |
| 45 | Displacement | /5IRD700/GGTGGTATCCCCAAGGGGACACTACGTACCGTGGTACGGC |
| | Primer | CGAGGTTGTG |
| 46 | 3T Incorporation | TCTCTATTGTGAGTCGTCTAGGTGGTATCCTTTTTTTTTTTTTTTTTTTT |
| | Template | |
| 47 | S1 Std Library | TTCTCATGTTTGACAGCTTAATTCCT-N40- |
| | | GGTGGTATCCCCAAGGGGAC |
| 48 | S1 Phe Library | TCATCGATAAGCTTTAATGCCACTCA-N40- |
| | | GGTGGTATCCCCAAGGGGAC |
| 49 | S1 Trp Library | TTGCTAACGCAGTCAGGCACCCAACA-N40- |
| | | GGTGGTATCCCCAAGGGGAC |
| 50 | TNFa Std Library | GGATCGTCAGTGCATTGAGAattcct-N40- |
| | | GGTGGTATCCCCAAGGGGAC |
| 51 | TNFa Phe Library | GGATCGTCAGTGCATTGAGAcactca-N40- |
| | | GGTGGTATCCCCAAGGGGAC |
| 52 | TNFa Trp Library | GGATCGTCAGTGCATTGAGAccaaca-N40- |
| | | GGTGGTATCCCCAAGGGGAC |
| 53 | Doped Trp-1 | TTGCTAACGCAGTCAGGCACTGGCCT-N′40- |
| | Library | GGTGGTATCCCCAAGGGGAC |

*Lower case letters denote barcode sequence of the std, phe, and trp libraries.
*/iSp9/ indicates a polyethylene glycol (PEG) spacer

### Library Synthesis

Three libraries were synthesized at the DNA level using a custom nucleotide distribution to minimize the occurrence of G-quadruplex structures. Each library contained a unique 6 nt barcode located between the forward primer and random region to signify the library chemotype (standard, Phe, and Trp). The initial libraries comprised an internal variable region of 40 nucleotides (30% A, 30% T, 20% G, 20% C) flanked on both sides by fixed-sequence primer binding sites. The libraries were purchased from the Keck Oligonucleotide Synthesis Facility (Yale University) and purified by 10% denaturing PAGE. The band corresponding to full-length product was excised, electroeluted, desalted, and quantified by UV absorbance. The phosphorylated DNA hairpin primer was ligated to the library by combining 5 nmol DNA library with 6 nmol of DNA hairpin in a final volume of 1 mL of 1×T4 DNA ligase buffer. The solution was denatured for 5 min at 95° C. and then annealed by incubating for 30 min at room temperature. Once annealed, 10,000 U of T4 DNA ligase was added and the reaction was incubated overnight at 24° C. The following day, the hairpin library was purified by 10% denaturing PAGE and quantified by UV absorbance.

The hairpin library was extended with tNTPs to form a chimeric TNA:DNA hairpin heteroduplex. In a 1 mL reaction volume containing 1 nmol hairpin library and 1× ThermoPol buffer was heated for 5 min at 95° C. to denature and then annealed by incubating for 30 min at 24° C. TNA polymerization was initiated by adding Kod RSGA (1 UM) and 100 M of each tNTP (tATP:tCTP:tGTP:tTTP or tUTP^{Phe} or tUTP^{Trp}) and incubating for 2 h at 55° C. Excess polymerase was extracted with phenol:chloroform:isoamyl alcohol (25:24:1, saturated with 10 mM Tris, pH 8.0, 1 mM EDTA). Following extraction, the TNA:DNA hairpin library was concentrated and the remaining tNTPs were removed by buffer exchange into water using a YM-30 Centricon filter device. Library was quantified by UV absorbance.

Next, the TNA strand was displaced by extending a DNA primer annealed to the hairpin loop with dNTPs. Accordingly, the TNA:DNA library (1 μM final) was resuspended in 1× ThermoPol buffer containing 500 UM dNTPs and 2 μM strand displacement primer. After heating for 5 min at 95° C. and slowly cooling to room temperature, the displacement reaction was initiated by adding Bst 3.0 DNA polymerase to a final concentration of 80 U/mL. The reaction was incubated for 1 h at 50° C. Bst polymerase was extracted with phenol:chloroform as described above. The TNA library was separated from the residual phenol and reaction components in a YM-30 Centricon. Any remaining phenol was removed by adding 900 μL of ethanol to the solution and drying completely. The libraries were resuspended in a selection buffer (S1: 150 mM NaCl, 25 mM Tris pH 8.0, TNFα: 20 mM imidazole, 450 mM NaCl, 20 mM Tris pH 8.0).

### Aptamer Selection

Aptamer selections were performed by passing each library through unmodified Ni-NTA agarose beads for 30 minutes at 24° C. with rotation to remove any sequences that bound to the affinity matrix. This step was performed for each round of selection. For the S1 selection, an additional negative selection step was performed with 1 μM hemagglutinin (HA) for 30 minutes at 24° C. with rotation, chosen as a generic viral coat protein. For this step, the flow through from the empty beads was passed through Ni-NTA beads containing the HA protein. The negative HA selection step was only performed in the first round of the S1 selection. The material collected from the flow-through of the negative selection was incubated with the appropriate His-tagged protein (either the S1 protein from SARS-CoV-2 or TNFα) poised at a concentration of 1 UM for 15 minutes at 24° C. with rotation. The mixture was incubated in a disposable plastic column with Ni-NTA beads for 15 minutes at 24° C. with rotation. The column was drained and washed three times with 400 μL of selection buffer to remove unbound and weakly bound molecules. A more stringent wash step was performed with 1 M NaCl to reduce the occurrence of nonspecific electrostatic interactions (S1: 1 M NaCl, 25 mM Tris pH 8.0, TNFα: 20 mM imidazole, 1 M NaCl, 20 mM Tris pH 8.0), followed by an additional wash with selection buffer to return to baseline salt concentrations before elution. Six 250 μL elutions were performed with elution buffer (S1: 500 mM imidazole, 150 mM NaCl, 25 mM Tris pH 8.0, TNFα: 100 mM imidazole, 450 mM NaCl, 20 mM Tris pH 8.0). Elution fractions were imaged on a LI-COR Odyssey CLx. The two elution fractions with the highest fluorescence were pooled and desalted using a NAP-5 DNA purification column (Cytiva, Marlborough, MA). TNA aptamers were amplified by PCR with library-specific PCR primers and purified using DNA Clean and Concentrator columns (Zymo Research, Irvine, CA). The single-stranded DNA library was regenerated through a second PCR using a PEGylated forward primer and the high fidelity PCRBIO HiFi polymerase, then purifying by 10% denaturing PAGE, cutting the corresponding band with a scalpel and recovering by electroelution.

### NGS Sequencing & Analysis

86 bp library members that remained in the pool after each round of selection were PCR amplified and purified as above. Amplicons were prepared as barcoded libraries and sequenced on a NovaSeq6000 with the S4 flow cell with the paired end setting for 200 cycles for an estimated 2-400 M reads per library, per round.

Sequencing data was parsed by filtering, trimming and aligning reads. Enrichment scores over the course of the selection were calculated for the top 10,000 most abundant sequences in the final round.

### Directed Evolution of Trp-1

A doped library of Trp-1 was synthesized at the DNA level with 15% doping of the other 3-nts at each position in the aptamer sequence. The DNA displayed TNA library was generated and taken through one round of selection exactly as described above with the exception that the S1 protein was poised at a concentration of 0.5 μM. TNA aptamers collected in the elution fractions were amplified by PCR with library-specific PCR primers, purified using DNA Clean and Concentrator columns (Zymo Research, Irvine, CA), and submitted for NGS analysis.

### Aptamer Preparation for Kinetic Analysis

Aptamers identified by sequencing were prepared as biotin-labeled TNA molecules using the PBS8 biotin 20mer DNA primer and the corresponding template. A 500 μL reaction volume containing 0.5 nmol of both primer and template as well as 1× ThermoPol Buffer was heated for 5 min at 95° C. to anneal. TNA polymerization was initiated by adding 100 UM of each tNTP and Kod RSGA polymerase (1 UM for standard bases or Phe, 2 UM for Trp) and then incubated for 2 h at 55° C. Full length biotin-labeled aptamers were purified by 10% denaturing polyacrylamide gel electrophoresis for 1.5 h at 18 W constant. TNA was recovered from the gel by electroelution and then buffer exchanged into water and concentrated using a YM-3 Centricon centrifugal filter device. TNA concentration was quantified by NanoDrop absorbance. Aptamers at a concentration of 100 nM were folded in BLI binding buffer (125 mM NaCl, 20 mM HEPES pH 7.5, 2 mM CaCl2, 0.05% Tween 20) by denaturing for 15 min at 95° C. and then cooling for 1 h at room temperature.

### BLI Screening

TNA aptamers were screened to identify sequences of interest for further characterization. Prior to testing, all sensors were equilibrated in BLI binding buffer for ≥1 h. Aptamers at a concentration of 100 nM were loaded on a single biosensor. A fixed target concentration of 100 nM for S1 and 1000 nM for TNFα was used. The BLI run was performed with the following steps: a buffer only baseline for 60 sec to equilibrate sensors, loading the aptamer for 200 sec, a second buffer only baseline for 200 sec, an association phase with the target protein for 600 sec for S1 or 120 sec for TNFα, and a dissociation phase for 600 sec for S1 or 120 sec for TNFα. The plate and reagents were incubated at 30° C. for S1 samples or 24° C. for TNFα for the duration of the experiment and for 10 min prior to each run. Data was analyzed using the Octet Data Analysis HT software. For screens, Savitzky-Golay filtering was applied before fitting both association and dissociation curves together to calculate approximate KD values.

### Full Kinetic Measurements for S1

Aptamer sequences of interest were characterized with four different concentrations of target and one buffer only sensor to determine background. Prior to testing, all sensors were equilibrated in BLI binding buffer (defined above) for ≥1 h. After aptamer folding, the BLI run was performed with the following steps: a buffer only baseline for 60 sec to equilibrate sensors, loading the aptamer for 200 sec, a second buffer only baseline for 200 sec, an association phase with the target protein for 600 sec for S1 or 120 sec for TNFα, and a dissociation phase for 600 sec for S1 or 120 sec for TNFα. The plate and reagents were incubated at 30° C. for S1 or 24° C. for TNFα for the duration of the experiment and for 10 min prior to each run. Data was analyzed using the Octet Data Analysis HT software. For full kinetics measurements, the buffer only baseline sample was used to subtract background from all samples before applying Savitzky-Golay filtering and fitting both the association and dissociation curves together and applying a global fit to determine KD and other metrics.

### Computational Method for Evolving Aptamers

A machine learning model based on the previously described Transformer architecture was trained using TensorFlow on the NGS data described above to predict the relative fraction of reads in the elution and flow-through pools from a TNA sequence. The Transformer encoder was trained with 8 attention heads per multiheaded layer and 3 encoder layers.

The model achieved a Most abundant versus Least abundant AUC of 0.69 and Most abundant vs Random AUC of 0.71 for the held out NGS test set. 10 k sequences were used for each of the Most abundant, Least abundant and Random pools.

Using this model, an in silico genetic algorithm was used to evolve a threomer with an estimated KD of 27 nM to the aforementioned threomer TNA 949 with a KD of 700±40 μM.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A based-modified xeno nucleic acid (XNA) nucleoside monomer comprising,
a) a synthetic, non-natural sugar,
b) a pyrimidine nucleotide base bound to the sugar moiety, wherein the pyrimidine nucleotide base comprises a chemical modification at position C-5 of the nucleobase, and
c) a phosphorus group bound to the sugar moiety.

2. The monomer of claim 1, wherein the synthetic, non-natural sugar is a threose sugar or a hexose sugar.

3. (canceled)

4. The monomer of claim 1, wherein the phosphorus group comprises a triphosphate group or a phosphoramidite group.

5. The monomer of claim 4, wherein the triphosphate group is bound to the C6′ position on a hexose sugar.

6. (canceled)

7. The monomer of claim 4, wherein the phosphoramidite group is bound to C3′ position on a hexose sugar.

8.-9. (canceled)

10. The monomer of claim 4, wherein the triphosphate group is bound to the C3′ position on a threose sugar.

11. (canceled)

12. The monomer of claim 4, wherein the phosphoramidite group is bound to the C2′ position of a threose sugar.

13. The monomer of claim 1, wherein the pyrimidine nucleotide base is bound to a C1′ position on the sugar moiety.

14. The monomer of claim 1, wherein the pyrimidine nucleotide base comprises a uracil base or a cysteine base.

15. The monomer of claim 1, wherein the chemical modification at position C-5 of the nucleobase are selected from a group consisting of phenylalanine side chain, a tryptophan side chain, a methyl side chain, a leucine side chain, a dioxol side chain, an isopentyl side chain, a dioxethyl side chain, a cyclopropyl side chain, a p-methoxy-phenyl side chain, a napthalene side chain, and a phenethyl side chain.

16.-28. (canceled)

29. A single-stranded xeno nucleic acid (XNA) aptamer for binding a target protein, the aptamer comprising one or more based-modified XNA nucleoside monomers comprising,
a) a synthetic, non-natural sugar,
b) a pyrimidine nucleotide base bound to the sugar moiety, wherein the pyrimidine nucleotide base comprises a chemical modification at position C-5 of the nucleobase, and
c) a phosphorus group bound to the sugar moiety.

30. The aptamer of claim 29, wherein the synthetic, non-natural sugar comprises a threose sugar or a hexose sugar.

31. The aptamer of claim 29, wherein the based-modified XNA nucleoside monomer comprises a modified threose nucleic acid (TNA) nucleoside monomer; wherein the modified TNA nucleoside monomer comprises a modified TNA triphosphate monomer.

32.-33. (canceled)

34. The aptamer of claim 29, wherein the phosphorus group comprises a triphosphate group or a phosphoramidite group.

35. The aptamer of claim 34, wherein the triphosphate group is bound to the C3′ position of the sugar moiety.

36.-37. (canceled)

38. The aptamer of claim 34, wherein the phosphoramidite group is bound to the C2′ position of the sugar moiety.

39. (canceled)

40. The aptamer of claim 29, wherein the pyrimidine nucleotide base is bound to the C1′ position on the sugar moiety; wherein the pyrimidine nucleotide base comprises an uracil residue, a cystine residue, or a combination thereof.

41. (canceled)

42. The aptamer of claim 29, wherein the chemical modification at position C-5 of the nucleobase are selected from a group consisting of phenylalanine side chain, a tryptophan side chain, a methyl side chain, a leucine side chain, a dioxol side chain, an isopentyl side chain, a dioxethyl side chain, a cyclopropyl side chain, a p-methoxy-phenyl side chain, a napthalene side chain, and a phenethyl side chain.

43. The aptamer of claim 29, wherein the aptamer comprises 20 to 40 based-modified XNA nucleoside monomers.

44. The aptamer of claim 29, wherein the target protein comprises a spike protein (S1), RBD (receptor binding domain) of S1, tumor necrosis factor (TNF)-α protein, HIV (human immunodeficiency virus) reverse transcriptase, human epidermal growth factor receptor 2 (HER2), trypsin, angiotensin-converting enzyme 2 (ACE2), or thrombin.

45.-54. (canceled)
